(19) United States
(12) Patent Application Publication
(10) Pub. No.: US 2023/0073248 A1
(43) Pub. Date: Mar. 9, 2023

(54) COMPOSITIONS AND METHODS FOR INDUCING IMMUNE TOLERANCE IN TRANSPLANTATION RECIPIENTS

(71) Applicants: The Regents of the University of Colorado. a body Corporate, Denver, CO (US); Angimmune LLC, Bethesda, MD (US); The General Hospital Corporation, Boston, MA (US)

(72) Inventors: Christene A. Huang, Denver, CO (US); David M. Neville, JR., Bethesda, MD (US); Elizabeth Anne Pomfret, Aurora, CO (US); Raimon Duran-Struuck, Bala Cynwyd, PA (US); Zhirui Wang, Denver, CO (US)

(21) Appl. No.: 17/820,808

(22) Filed: Aug. 18, 2022

Related U.S. Application Data
(63) Continuation of application No. PCT/US2021/018607, filed on Feb. 18, 2021.
(60) Provisional application No. 62/978,273, filed on Feb. 18, 2020.

(51) Int. Cl. A61K 39/395; A61K 35/14; A61K 38/45; A61K 31/436; A61K 31/5377; A61K 31/573; A61K 31/58; A61K 31/519; A61K 38/13; A61K 31/52; A61K 31/42; A61P 37/06

(52) U.S. Cl. CPC A61K 39/3955; A61K 35/14; A61K 38/45; A61K 31/436; A61K 31/5377; A61K 31/573; A61K 31/58; A61K 31/519; A61K 38/13; A61K 31/52; A61K 31/42; A61P 37/06

(57) ABSTRACT

Embodiments disclosed herein relate to compositions and methods for inducing transplantation tolerance using immunomodulation agents. In certain embodiments compositions and methods disclosed herein, concern administering a composition including, but not limited to, anti-CD3 immunotoxin and administering a composition including, but not limited to, peripheral blood cells obtained from a donor of an organ, tissue or cells to be transplanted. In some embodiments, compositions and methods disclosed here can be used for modulating B- and/or T-cell-mediated immunity and/or rejection by reducing or eliminating anti-donor antibody production. Other embodiments concern modulating T-cell production in a subject preparing for, undergoing organ, tissue or cellular transplantation; or having or expected of developing GvHD for reducing the risk of, preventing or treating rejection or GvHD. In certain embodiments, combination compositions of anti-CD3 immunotoxin and peripheral blood cells from a donor are contemplated.

### PRIORITY

This U.S. Continuation application claims the benefit of International Application PCT/US202/018607, filed on Feb. 18, 2021, which claims the benefit of priority pursuant to 35 U.S.C. § 119(e) to U.S. Provisional Application No. 62/978,273 filed on Feb. 18, 2020. These applications are incorporated herein by reference in their entirety for all purposes.

### GOVERNMENT FUNDING

This invention was made with government support under grant numbers 1-R21 AI115136-01A1; 1R56AI121254-01, 1R01A184657-01, 5P01CA111519-04 awarded by National Institutes of Health to Dr. Huang and grant number 1K01RR024466 awarded to Dr. Duran-Struuck. The government has certain rights in the invention.

### FIELD

Embodiments disclosed herein relate to compositions and methods for inducing organ, tissue or cellular transplantation tolerance using immunomodulation agents. In some embodiments, compositions and method disclosed herein concern administering a composition including, but not limited to, anti-CD3 immunotoxin to a subject having a transplantation or implantation to improve tolerance to the transplantation or implantation. In certain embodiments, compositions and methods disclosed herein concern administering a composition including, but not limited to, anti-CD3 immunotoxin and administering a composition including, but not limited to, peripheral blood cells obtained from a donor of an organ, tissue or cells to be transplanted. In some embodiments, compositions and methods disclosed here can be used for modulating B cell and/or T-cell-mediated immunity and/or rejection by reducing or eliminating allo- and/or anti-donor antibody production and/or donor reactive T-cell production in a subject preparing for, or undergoing organ, tissue or cellular transplantation. In certain embodiments, combination compositions of anti-CD3 immunotoxin and peripheral blood cells from a donor are contemplated. In certain embodiments, compositions including, but not limited to, anti-CD3 immunotoxins can be used to reduce the onset of, transplantation rejection or graft versus host (GvHD) disease, or treat rejection or GvHD in a subject.

### BACKGROUND

Certain conditions such as antibody mediated rejection (AMR) can lead to graft loss after transplantation or implantation and other complications. AMR is considered a leading cause of graft loss after kidney transplantation. The development of de novo donor specific antibody (DSA) post transplantation is associated with poor graft outcomes including late acute antibody-mediated rejection, chronic antibody-mediated rejection, and transplant glomerulopathy. In addition, severe limitations of organ donor pools lead to long waiting lists for life-saving organs for patients with end-stage renal, heart, liver, lung, pancreas, bowel disease and other conditions. With improvements in transplant techniques and immunosuppression (IS) regimens, short-term kidney transplant survival rate has improved significantly with about a 90% survival at 1 year. However, long-term outcome has not markedly improved in the same time period, with 70-80% kidney graft survival at 5 years and approximately 50% graft loss by 10-12 years. It has been demonstrated that short-term and long-term kidney graft attrition are two separate processes whose evolution is not necessarily interrelated. Antibody mediated rejection (AMR) is an important barrier to long-term success after renal transplantation, and late antibody-mediated rejection is thought to be one of the leading causes for graft loss. Outcomes for other organs are generally worse but follow similar trends. For example, an alloimmune response against polymorphic human leukocyte antigens (HLA) is considered a leading cause of both hyperacute and chronic rejection of allografts. Additional threats to allografts are non-HLA directed immune responses and non-immunologic factors like ischemia reperfusion injury. While cell mediated rejection can be controlled with immunosuppression (IS), humoral immunity is only indirectly targeted by IS and therefore, remains mostly resistant to treatment. In addition, alloantibodies can interfere with the induction of transplantation tolerance. Therefore, there is a need for therapies to condition transplant recipients to reduce recipient immunosuppressive conditioning, avoid antibody-mediated rejection (AMR), and improve outcomes.

### SUMMARY

Embodiments of the instant disclosure relate to compositions and methods for inducing organ, tissue and/or cellular transplantation tolerance in a subject in need thereof. In some embodiments, compositions and methods disclosed here can be used for modulating B cell- and/or T-cell-mediated immunity and/or rejection by reducing or eliminating allo- and/or anti-donor antibody production and/or T-cell production in a subject preparing for, or undergoing organ, tissue or cellular transplantation. In certain embodiments, compositions and methods disclosed herein concern inducing immune tolerance by reducing or eliminating allo-antibody responses to allografts received from a donor. In accordance with these embodiments, the donor can be a major histocompatibility complex (MHC) fully matched, a partial MHC matched (e.g. haplo-mismatched) or a fully MHC mismatched donor compared to the recipient receiving the transplant. In other embodiments, the subject can be a human subject and compositions and methods disclosed herein can be used to induce transplant or infusion tolerance from a donor as a human leukocyte antigen (HLA) matched, a partial HLA matched or an HLA fully mismatched human donor compared to the human recipient receiving the transplant or infusion. In accordance with these embodiments, to reduce or eliminate allograft rejection, a subject scheduled for, or having an allograft transplantation or cellular infusion can be administered a composition including, but not limited to, one or more anti-CD3 immunotoxin (CD3 immunotoxin-based conditioning) and administered a composition including, but not limited to, peripheral blood cells obtained from the donor of the donor organ, tissue or cells and inducing allograft tolerance in the recipient subject to the allograft. In other embodiments, combination compositions of anti-CD3 immunotoxin and peripheral blood cells obtained from the donor of the donor organ, tissue or cells can be administered to a subject recipient scheduled for, or having a transplantation or cellular infusion. These compositions and methods prolong graft survival by inducing tolerance in the subject recipient and improving outcome and long-term outcome of the subject receiving such a transplantation by inducing tolerance and reducing or eliminating antibody-mediated response (AMR) to the donor graft and/or T-cell mediated immunities.

In certain embodiments, these combination compositions and/or methods disclosed herein improve allograft tolerance without the need for hematopoietic stem cell (HCT) engraftment. In other embodiments, these combination compositions and/or methods improve allograft survival without immunosuppression (IS); for example, without the need for treatment using immunosuppressive agents or irradiation or other technique to reduce or eliminate the subject's immune system prior to, during or after transplantation or implantation. In yet other embodiments, these combination compositions and/or methods improve allograft tolerance without the need for hematopoietic stem cell (HCT) engraftment and without immunosuppression (IS); for example, without the need for treatment using immunosuppressive agents or irradiation or other technique to reduce or eliminate the subject's immune system and without stem cell engraftment. In other embodiments, these combination compositions and/or methods can further include administering an immunosuppressant to the subject depending on need for improving transplantation outcomes. In some embodiments, the combination composition or individual compositions can be administered to a subject before, during and/or after allograft transplantation or implantation.

In some embodiments, a subject contemplated herein can be a subject scheduled for or undergoing an organ, tissue or cellular transplantation or implantation obtained from a donor, an allograft. In accordance with these embodiments, a donor organ, tissues or cells can include, but is not limited to, kidney, heart, lung, liver, intestine, pancreas, skin, eye, vascular composite allografts (VCAs), leukocytes, hepatocytes, pancreatic islets, bone marrow, corneal epithelial cells, or other transplant from a donor or a combination thereof.

In certain embodiments, the subject scheduled for or undergoing transplantation can receive compositions for reducing B-cell or B-cell related responses disclosed herein as well as receiving compositions for reducing or eliminating T-cell or T-cell responses; for example, by repressing both B-cells and T-cells related activities and then transplanting an allograft in the subject. In certain embodiments, repression of B-cells and/or T-cells can be transient or prolonged depending on need. In some embodiments, compositions and methods disclosed herein can be used to reduce or eliminate AMR and/or modulate T-cells for a period of time while maintaining the immune system of the subject to fight infection and other conditions. In accordance with these embodiments, the donor organ, tissue and/or cells to be transplanted can be transplanted in the subject recipient at the time of providing donor peripheral blood cells to the subject or after. In some embodiments, the donor organ, tissue and/or cells to be transplanted can be transplanted in the subject recipient after donor peripheral blood cells are administered immediately after, within one hour after, hours after, about a day after, about 2 days after, about 1 week after, about 2 weeks after, about 3 weeks after, up to about 10 weeks after.

In other embodiments, compositions and methods are disclosed for modulating or depleting T cells in a subject scheduled for, or undergoing or having undergone transplantation or implantation. In some embodiments, a compositions and methods disclosed herein concern reducing transplantation rejection before, during or after transplantation; for example, by administering to a subject scheduled for or undergoing a transplantation, a composition including, but not limited to, one or more anti-CD3 immunotoxin and preventing or reducing transplantation rejection in the subject. In certain embodiments, compositions including, but not limited to, one or more anti-CD3 immunotoxin can be used to treat an acute T-cell mediated rejection episode in a subject having received a transplanted organ, tissue and/or cells to reduce or prevent rejection in the subject and improve transplantation outcome.

In certain embodiments, a subject contemplated herein is at risk of developing or has graft versus host disease (GvHD). In accordance with these embodiments, a subject can be administered a composition including, but not limited to, one or more anti-CD3 immunotoxin to reduce onset of, or treat GvHD in the subject. In other embodiments, the subject has acute, chronic or steroid-refractory GvHD and compositions and methods disclosed herein treat the acute, chronic or steroid-refractory GvHD; for example, by modulating T-cell populations in the subject. In accordance with these embodiments, immunosuppressive agents can be reduced and/or stopped altogether in a subject having or suspected of developing GvHD. In other embodiments, immunosuppressive agent treatment regimens can be modified in the subject.

In certain embodiments, the one or more anti-CD3 immunotoxin can be generated using recombinant technologies. In some embodiments, the one or more anti-CD3 immunotoxin can be a fusion molecule. In other embodiments, the one or more anti-CD3 immunotoxin can be a fusion molecule for use in humans.

In certain embodiments, a combination composition including, but not limited to, one or more anti-CD3 immunotoxin and peripheral blood cells from the donor of the transplanted organ, tissue or cells are contemplated. In certain embodiments, the one or more anti-CD3 immunotoxin of the compositions can be a construct created by recombinant technologies. In other embodiments, the one or more anti-CD3 immunotoxin can be a fusion molecule and can further be a fusion molecule designed for the subject being treated (e.g. human subject). In certain embodiments, donor peripheral blood cells can include, but are not limited to, peripheral blood mononuclear cells (PBMC).

In some embodiments, the subject is scheduled for or undergoing or has had a solid organ transplantation. In certain embodiments, the subject is preparing for, undergoing or has had a kidney transplant. In other embodiments, the donor kidney can be from an MHC fully matched, a partial MHC matched (e.g. haplo-mismatched) or a fully MHC mismatched donor compared to the subject receiving the transplant. In accordance with these embodiments, the subject can be treated with combination therapies disclosed herein or a composition including, but not limited to, one or more anti-CD3 immunotoxin.

In certain embodiments, compositions and methods disclosed herein can be used for preparing a subject for organ, tissue or cellular transplantation. In some embodiments, compositions and methods disclosed herein can be used to reduce transplant rejection by inducing tolerance in the subject and prolong transplant survival.

In certain embodiments, kits are contemplated. In accordance with these embodiments, a kit can include one or more anti-CD3 immunotoxin and peripheral blood cells from the donor; and at least one container. In other embodiments, kits can further include one or more delivery device. In yet other embodiments, kits can include devices for obtaining peripheral blood cells from the donor.

### BRIEF DESCRIPTION OF THE DRAWINGS

FIGS. 1A-1B illustrate tables outlining exemplary experimental designs for testing immune tolerance in an animal model in accordance with some embodiments of the present disclosure.

FIGS. 2A-2G represent grafts illustrating immunophenotyping pre- and post-anti-CD3 immunotoxin-based conditioning (e.g. ITC) in accordance with some embodiments of the present disclosure.

FIGS. 3A-3B illustrate (A) a plot demonstrating transient donor chimerism and (B) lack of persistent donor engraftment of blood and various tissues in accordance with some embodiments of the present disclosure.

FIGS. 4A-4F illustrate examples of normal antibody response to subcutaneous challenge across haplo and full MHC mismatched donors in accordance with certain embodiments of the present disclosure.

FIGS. 5A-5E illustrate by graphical representation lack of antibody response following compositions and methods to induce tolerance in a subject recipient in accordance with certain embodiments of the present disclosure.

FIGS. 6A-6B illustrates by graphical representation (A) and in a table (B) immune competence in animals unresponsive to donor challenge in accordance with certain embodiments of the present disclosure.

FIGS. 7A-7I illustrate histology of draining lymph nodes in accordance with certain embodiments of the present disclosure.

FIGS. 8A-8B illustrate an enhanced view of certain panels in FIG. 7 representing histology of draining lymph nodes in accordance with certain embodiments of the present disclosure.

FIG. 9 is a table representing analysis of immune cell absence or presence in various treatments in accordance with certain embodiments of the present disclosure.

FIG. 10 illustrates flow cytometry images representing percentages of certain peripheral blood B cells analyzed with and without ITC conditioning in accordance with certain embodiments of the present disclosure.

FIGS. 11A-11C represent data collected from an ITC conditioned animal model after challenges with donor cells both intravenous and subcutaneous (A); analysis of cytotoxic anti-donor antibody (B) during the course of study in A compared to normal antibody responses over a predetermined time period (C) in accordance with certain embodiments of the present disclosure.

FIGS. 12A-12D illustrate analysis of graft acceptance in an animal model after anti-CD3 immunotoxin conditioning and peripheral blood donor cell infusion where A-C illustrate various parameters to assess immune status and organ function and D represents a histological image of the transplant post transplantation in accordance with certain embodiments of the present disclosure.

FIG. 13 illustrates a histological stained image comparing anti-thymocyte globulin (rATG) with CD3 immunotoxin (CD3 IT) treatments in an animal model where lymph node sections were stained for analysis of T-cell and B-cell infiltration pre- and post-treatment in accordance with certain embodiments of the present disclosure.

### DEFINITIONS

Terms, unless specifically defined herein, have meanings as commonly understood by a person of ordinary skill in the art relevant to certain embodiments disclosed herein or as applicable.

Unless otherwise indicated, all numbers expressing quantities of agents and/or compounds, properties such as molecular weights, reaction conditions, and as disclosed herein are contemplated as being modified in all instances by the term “about.” Accordingly, unless indicated to the contrary, the numerical parameters in the specification and claims are approximations that can vary from about 10% to about 15% plus and/or minus depending upon the desired properties sought as disclosed herein. Numerical values as represented herein inherently contain standard deviations that necessarily result from the errors found in the numerical value's testing measurements.

As used herein, the term “subject,” “subject recipient” and “patient” are used interchangeably herein and refer to both human and nonhuman animals. The term “nonhuman animals” of the disclosure includes all vertebrates, e.g., mammals and non-mammals, such as nonhuman primates, sheep, dog, cat, horse, cow, chickens, amphibians, reptiles, and the like. In some embodiments, the subject can be a human such as an adult, child, adolescent or infant.

As used herein, “treatment,” “therapy” “treatment regimen” and/or “therapy regimen” can refer to the clinical intervention made in response to a disease, disorder or physiological condition manifested by a subject or to which a subject can be susceptible. The aim of treatment includes the alleviation or prevention of symptoms, slowing or stopping the progression or worsening of a disease, disorder, or condition and/or the remission of the disease, disorder or condition. As used herein, the term “treating” can refer to the application or administration of a composition including one or more active agents to a subject, who has a target disease or disorder, a symptom of the disease/disorder, or a predisposition toward the disease/disorder, with the purpose to cure, heal, alleviate, relieve, alter, remedy, ameliorate, improve, or affect the disorder, a symptom of the disease or disorder, or the predisposition toward the disease or disorder.

As used herein, “prevent” or “prevention” refers to eliminating or delaying the onset of a condition, disorder, disease or physiological condition, or to the reduction of the degree of severity of a condition, disorder, disease or physiological condition, relative to the time and/or degree of onset or severity in the absence of intervention.

The term “effective amount” or “therapeutically effective amount” refers to an amount sufficient to effect beneficial or desirable biological and/or clinical results.

Alleviating a target disease/disorder or condition includes delaying the development or progression of the disease, or reducing disease severity or prolonging survival. Alleviating the disease or prolonging survival does not necessarily require curative results. As used therein, “delaying” the development of a target disease, condition or disorder can mean to defer, hinder, slow, retard, stabilize, and/or postpone progression of the disease. This delay can be of varying lengths of time, depending on the history of the disease and/or individuals being treated. A method that “delays” or alleviates the development of a disease, or delays the onset of the disease, is a method that reduces probability of developing one or more symptoms of the disease in a given time frame and/or reduces extent of the symptoms in a given time frame, when compared to not using the method. Such comparisons are typically based on clinical studies, using a number of subjects sufficient to give a statistically significant result.

As used herein the term “detectable label” can refer to any moiety that generates a measurable signal via optical, electrical, or other physical indication of a change of state of a molecule or molecules coupled to the moiety. Such physical indicators encompass spectroscopic, photochemical, biochemical, immunochemical, electromagnetic, radiochemical, and chemical means, such as but not limited to fluorescence, chemifluorescence, chemiluminescence, and the like. As used with reference to a labeled detection agent, a “direct label” is a detectable label that is attached, by any means, to the detection agent. As used with reference to a labeled detection agent, an “indirect label” is a detectable label that specifically binds the detection agent. Thus, an indirect label includes a moiety that is the specific binding partner of a moiety of the detection agent. Biotin and avidin are examples of such moieties that are employed, for example, by contacting a biotinylated antibody with labeled avidin to produce an indirectly labeled antibody. An indicator reagent can be used to contact a detectable label to produce a detectable signal. In certain embodiments, samples of a subject recipient can be analyzed for donor specific antibodies using any assay known in the art.

As used herein, the term “sample” generally refers to a biological material obtained from a subject or donor. The biological material can be derived from any biological source but preferably is a biological fluid likely to contain the target analyte. Examples of biological materials can include, but are not limited to, stool, whole blood, serum, plasma, red blood cells, platelets, bronchial lavage, bone marrow aspirate, pleural effusion, interstitial fluid, saliva, ocular lens fluid, cerebrospinal fluid, sweat, urine, ascites fluid, mucous, nasal fluid, sputum, synovial fluid, peritoneal fluid, vaginal fluid, menses, amniotic fluid, semen, as well as tumor tissue or any other bodily constituent or any tissue culture supernatant that could contain the analyte of interest. Samples herein can be obtained by routine procedures such as but not limited to venipuncture, tissue biopsy including needle biopsy, swab, wipe, and fluid collection. Samples herein are obtained from an animal, preferably a mammal, and more preferably a human. The sample can be used directly as obtained from the biological source or following a pretreatment to modify the character of the sample. For example, such pretreatment can include preparing plasma from blood, diluting viscous fluids and so forth. Methods of pretreatment can also involve centrifugation, filtration, precipitation, dilution, distillation, mixing, concentration, inactivation of interfering components, the addition of reagents, lysing, etc. If such methods of pretreatment are employed with respect to the test sample, such pretreatment methods are such that the target analyte remains in the test sample at a concentration proportional to that in an untreated test sample (e.g., namely, a test sample that is not subjected to any such pretreatment method(s)).

Unless otherwise defined, all technical terms used herein have the same meaning as commonly understood by one of ordinary skill in the art to which this disclosure belongs.

### DETAILED DESCRIPTION OF THE INVENTION

In the following sections, certain exemplary compositions and methods are described in order to detail certain embodiments of the invention. It will be obvious to one skilled in the art that practicing the certain embodiments does not require the employment of all or even some of the specific details outlined herein, but rather that concentrations, times and other specific details can be modified through routine experimentation. In some cases, well known methods, or components have not been included in the description.

Embodiments of the instant disclosure relate to compositions and methods for inducing organ, tissue and/or cellular transplantation tolerance in a subject in need thereof. In some embodiments, compositions and methods disclosed here can be used for modulating B cell- and/or T-cell-mediated immunity and/or rejection by reducing or eliminating allo- and/or anti-donor antibody production and/or T-cell production in organs and in tissues in a subject preparing for, undergoing or having undergone organ, tissue or cellular transplantation. In certain embodiments, compositions and method disclosed herein concern inducing immune tolerance by reducing or eliminating allo-antibody production and responses to allografts received from a donor. In accordance with these embodiments, the donor can be a major histocompatibility complex (MHC) fully matched, a partial MHC matched (e.g. haplo-mismatched) or a fully MHC mismatched donor compared to the recipient receiving the transplant where compositions and methods disclosed herein reduce or eliminate allo-antibody production. In other embodiments, the subject can be a human subject and compositions and methods disclosed herein can be used to induce transplant or infusion tolerance from a donor as a human leukocyte antigen (HLA) matched, a partial HLA matched or an HLA fully mismatched human donor compared to the human recipient receiving the transplant or infusion. In accordance with these embodiments, to reduce or eliminate allo-antibody production in allograft transplantation, a subject scheduled for, or having an allograft transplantation or cellular infusion from a donor, the subject can be administered a composition including, but not limited to, one or more anti-CD3 immunotoxin (e.g. immunotoxin-based conditioning) and administered a composition including, but not limited to, peripheral blood cells obtained from the donor of the donor organ, tissue or cells and inducing allograft tolerance in the recipient subject to the allograft, tissue or cells. In other embodiments, combination compositions of anti-CD3 immunotoxin and peripheral blood cells obtained from the donor of the donor organ, tissue or cells can be administered to a subject recipient scheduled for or having a transplantation or cellular infusion. These compositions and methods prolong graft survival and improve outcome of the subject receiving such a transplantation by inducing tolerance and reducing or eliminating antibody-mediated response (AMR) to the donor graft and/or T-cell mediated immunities related to graft rejection and/or rejections by the graft (e.g. GvHD). In some embodiments, a subject recipient having GvHD has undergone bone marrow or other implantation or experienced another condition resulting in GvHD contemplated to be treated by anti-CD3 immunotoxin or other immunotoxin therapies to alleviate the symptoms of GvHD and/or GvHD in the subject recipient.

In certain embodiments, T-cell production in organs and tissues can be transiently depleted. In other embodiments, B-cell repression and allo-antibody suppression can be prolonged over a period of time or permanently. In other embodiments, compositions and methods disclosed herein can be used to suppress or eliminate B-cell and/or T-cell activity for about 24 hours to about 7 weeks after administration of peripheral donor blood cells. In certain embodiments, T-cell depletion can be depleted for periods of a year or more, or about 9 months or about 6 months or less. In some embodiments, CD4+ T cells can be depleted for about 6 to about 9 months due to reconstitution in for example, the thymus, while other T cells such as other CD3+ T cell subsets can be depleted for shorter periods than the CD4+ T cells. In accordance with these embodiments, more than one transplantation can occur with respect to tissue or cell transplantation if the donor is the same donor. Alternatively, donor peripheral blood cells can be obtained from a second, third or fourth donor, etc. if needed when an organ, tissue or cells are from more than one donor and administered to the subject recipient before or during transplantation or subsequent transplantation events.

In certain embodiments, these combination compositions and/or methods disclosed herein can improve allograft tolerance without the need for hematopoietic stem cell (HCT) engraftment. In some embodiments, donor peripheral blood cells can be obtained from the donor collected by any method known in the art (e.g. apheresis) and in certain embodiments, the donor peripheral blood cells or donor hematopoietic cells can be obtained without the need for stem cell enrichment. In certain embodiments, donor peripheral blood cells obtained from the donor of use in a subject recipient does not require or include stem cell engraftment which can reduce risk of developing GvHD or other complications in the subject recipient.

In some embodiments, transient or long-term or intermittent immunosuppressive treatment methods can be included in compositions and methods disclosed herein to reduce transplant rejection or improve transplantation tolerance in a subject in conjunction with compositions and methods disclosed herein (e.g. irradiation, immunosuppressive treatment regimens). Non-limiting examples of immunosuppressive treatment regimens can include calcineurin inhibitors (e.g., tacrolimus, cyclosporine, pimecrolimus), antiproliferative agents (e.g., mycophenolate mofetil, mycophenolate sodium, azathioprine), nTOR inhibitors (e.g., sirolimus, everolimius), steroids (e.g., corticosteroids, prednisone), depleting antibodies (e.g., antithymocyte globulin, alemtuzumab, rituximab) non-depleting antibodies (e.g., basiliximab, daclizumab), belatacept, and the like. In other embodiments, these combination compositions and/or methods improve allograft survival without immunosuppression (IS); for example, without the need for treatment using immunosuppressive agents or irradiation or other technique to reduce or eliminate the subject's immune system prior to, during or after transplantation or implantation. In yet other embodiments, these combination compositions and/or methods improve allograft tolerance without the need for hematopoietic stem cell (HCT) engraftment and without immunosuppression (IS); for example, without the need for treatment using immunosuppressive agents or irradiation or other technique to reduce or eliminate the subject's immune system and without stem cell engraftment. In other embodiments, these combination compositions and/or methods can further include administering an immunosuppressant to the subject depending on need for improving transplantation outcomes. In some embodiments, the combination composition or individual compositions can be administered to a subject before, during and/or after allograft transplantation or implantation.

In some embodiments, a subject contemplated herein can be a subject scheduled for or undergoing an organ, tissue or cellular transplantation or implantation obtained from a donor, an allograft. In accordance with these embodiments, a donor organ, tissues or cells can include, but is not limited to, kidney, heart, lung, liver, intestine, pancreas, skin, eye, vascular composite allografts (VCAs), leukocytes, hepatocytes, pancreatic islets, bone marrow, corneal epithelial cells, or other donor transplant or a combination thereof. In some embodiments, transplanted tissue can be a vascularized composite allograft (e.g., allografts such as face, hand, or a tissue transplant that includes multiple tissue types).

In some embodiments, the subject recipient is scheduled for or undergoing a kidney transplantation where the donor is a haplo-identical, haplo-mismatched or fully-mismatched MHC. In accordance with these embodiments, the subject recipient of the kidney can be treated before, during or after transplantation with anti-CD3 immunotoxin and further treated before, during or after with peripheral blood cells obtained from the donor of the donor kidney. In other embodiments, compositions and methods disclosed herein reduce or eliminate development of de novo donor specific antibody (DSA) post transplantation reducing or eliminating late acute antibody-mediated rejection, chronic antibody-mediated rejection, and transplant glomerulopathy for improved transplant outcomes. In certain embodiments, the subject recipient has chronic kidney disease (CKD). As contemplated herein, CKD encompasses five stages of disease severity wherein disease severity can be measured by a subjects estimated glomerular filtration rate (eGFR). As contemplated herein, stage I CKD can be classified as a subject having an eGFR of 90 ml/minute or greater; stage II CKD can be classified as a subject having an eGFR between 60 and 89 ml/minute; stage III CKD can be classified as a subject having an eGFR between 30 and 59 ml/minute; stage IV CKD can be classified as a subject having an eGFR between 15 and 29 ml/minute; and stage V CKD can be classified as a subject having an eGFR of less than 15 ml/minute. In certain embodiments, the subject recipient has stage V kidney disease (CKD). In other embodiments, the subject recipient has end stage renal disease (ESRD).

In some embodiments, improved transplant outcome by prolonged transplant tolerance can relieve burden regarding limitations of organ donor pools, reduce waiting lists for life-saving organs for subject recipients with end-stage renal, heart, liver, lung, pancreas, bowel conditions and other conditions. In yet other embodiments, improvements in transplant protocols as disclosed herein for long-term outcome can have an overarching effect on the backlog of transplant recipients. In some embodiments, compositions and methods disclosed herein improve tolerance by a recipient subject of fully MHC mismatched organ, tissue and cell implantation by; for example, reducing or eliminating antibody mediated rejection (AMR) and late antibody-mediated rejection for improved graft survival.

Other embodiments disclosed herein concern combination therapies where treatment with compositions including one or more anti-CD3 immunotoxin and/or donor peripheral blood cells can be combined with other known treatments such as treatment for reducing or eliminating ischemia reperfusion injury (I/R). In some embodiments, agents of use to reduce or treat I/R can include anti-inflammatory agents such as alpha-1 antitrypsin, metabolic techniques, revascularization techniques, and/or restorative infusion of oxygen and other gases to the organ to reduce or eliminated I/R and I/R side effects in a subject.

In some embodiments, the donor peripheral blood cells can be administered to a transplant subject by intravenous infusion; for example, but not limited to, infusion through a portal vein or by other infusion such as a renal artery or the like. In some embodiments, anti-CD3 immunotoxin and donor peripheral blood cells can be co-administered by the same or different methods, at the same time or sequentially. In other embodiments, anti-donor antibody responses can be measured in a subject recipient after receiving the donor peripheral blood cells in order to assess whether additional infusions may be needed. Anti-donor antibody concentrations or levels in a subject recipient sample can be measured by any method known in the art. In certain embodiments, samples can be any body fluid such as blood or serum obtained from the subject recipient for analyzing for presence or level of anti-donor antibodies. In some embodiments, administering peripheral donor blood cells to a subject recipient requires little to no engraftment. In some embodiments, peripheral donor blood cells can be obtained from the donor without cytokine mobilization. In other embodiments, administration of donor peripheral blood cells can be by infusion or other method where the peripheral blood cells can be collected from the donor over a period of time collecting about 1 million to about 1 billion donor cells/kg using any technique known in the art (e.g. leukapheresis) for use in a subject disclosed herein. In other embodiments, about 0.5 million to about 200 million donor cells/kg can be collected (and used) and include unselected donor peripheral blood cells; for example, any donor peripheral blood cells that are not enriched or selected and do not require enriched CD4+ cell populations or other selective measures such as stem cell selectivity or enrichment. In accordance with these embodiments, donor peripheral blood cells can be collected without selection or enrichment unlike other cellular implantation techniques known in the art for use in compositions and methods disclosed herein. In some embodiments, the collected donor peripheral blood cells can be immediately administered to the subject recipient. In other embodiments, the collected donor peripheral blood cells can be stored for later use. In yet other embodiments, the collected donor peripheral blood cells can be administered to the subject recipient in combination with, at the same time or after anti-CD3 immunotoxin is provided to the subject recipient. In accordance with these embodiments, the collected donor peripheral blood cells can be administered to the subject recipient (e.g. about 0.5 million to about 1 billion donor cells/kg) about 30 minutes, to about 45 minutes, to about 1 hour, to about 2 hours, to about 5 hours or more after the subject recipient receives one or more doses of anti-CD3 immunotoxin before, during or after the subject recipient receives the transplanted organ, tissue and/or cells. In certain embodiments, it is contemplated that compositions and methods disclosed herein can dramatically reduce or elimination humoral donor reactivity for more than 3 months, for more than 6 months, for more than 9 months, for about 1 year, 2 years, 3 years, 4 years, 5 years, 6 years, 7 years, 8 years, 9 years, 10 years, 15 years, 20 years, or more. It is noted that humoral unresponsiveness in embodiments of the instant disclosure can be donor-specific as challenges with other immunogen mount an antibody response; therefore, permitting the subject recipient's B-cell responses to react normally if confronted with infectious agents or other conditions requiring a stable immune reaction.

In other embodiments, compositions and methods disclosed herein reduce or eliminate the need for using immunosuppressive agents to control immune responses in the subject recipient. In some embodiments, one or more immunosuppressant agents can be administered to the subject as needed to control aberrant immune responses in the subject recipient. In accordance with these embodiments, immunosuppressant agents can include, but are not limited to, one or more of a steroid, Janus kinase inhibitors, calcineurin inhibitors, mTOR inhibitors, IMDH inhibitors, polyclonal antibodies, and monoclonal antibodies. In accordance with these embodiments, immunosuppressant agents can include, but are not limited to, one or more of prednisone (e.g. Deltasone, Orasone), budesonide (Entocort EC), prednisolone (Millipred), tofacitinib (Xeljanz), cyclosporine (Neoral, Sandimmune, SangCya), tacrolimus (Astagraf XL, Envarsus XR, Prograf), sirolimus (Rapamune), everolimus (Afinitor, Zortress), azathioprine (Azasan, Imuran), leflunomide (Arava), mycophenolate (CellCept, Myfortic), abatacept (Orencia), adalimumab (Humira), anakinra (Kineret), certolizumab (Cimzia), etanercept (Enbrel), golimumab (Simponi), infliximab (Remicade), ixekizumab (Taltz), natalizumab (Tysabri), rituximab (Rituxan), secukinumab (Cosentyx), tocilizumab (Actemra), ustekinumab (Stelara), vedolizumab (Entyvio), basiliximab (Simulect), daclizumab or (Zinbryta) or other immunosuppressant of use depending on the subject to be transplanted and the transplant. In certain embodiments, a subject recipient can be treated with an immunosuppressant before, during or after compositions and methods of improving graft tolerance are provided to a subject recipient. In some embodiments, a subject recipient can be pre-treated with an immunosuppressant agent. In accordance with these embodiments, a subject can be treated with cyclosporine or similar agent; for example, about a few days, a week to a few weeks before treatment and subsequent transplantation disclosed herein at an acceptable concentration (e.g. about 100 to about 350 ng/ml). In some embodiments, immunosuppressive treatment can continue after transplantation for about 1 year or more and can be discontinued depending on status of transplant and presence of donor specific antibodies, for example.

In certain embodiments, the subject scheduled for or undergoing transplantation can receive compositions for reducing B-cell or B-cell related responses disclosed herein as well as receiving compositions for reducing or eliminating T-cell or T-cell responses; for example, by repressing both B-cells and T-cells related activities and then transplanting an allograft in the subject recipient. In certain embodiments, repression of B-cells and/or T-cells can be transient or prolonged depending on need. In some embodiments, compositions and methods disclosed herein can be used to reduce or eliminate AMR and/or modulate T-cells for a period of time while maintaining the immune system of the subject to fight infection and other conditions. In accordance with these embodiments, B cell responses in a subject recipient receiving such a treatment can recover while eliminating AMR in the subject recipient for at least one week, at least two weeks, at least one month, at least 3 months or longer allowing the subject recipient B cell responses to recover and remain immunocompetent having normal B cell responses after a period of time (e.g. 1 to 6 months after treatment). In some embodiments, AMR can be permanently eliminated in the subject to prolong graft survival. In other embodiments, T-cell depletion can be transient where a subject recipient of an organ, tissue or cell transplantation can be treated with anti-CD3 immunotoxin to eliminate T-cells for about 1 week, to about 2 weeks, to about 3 weeks, to about 4 weeks, to about 2 months, to about 4 months, to about 6 months, to about 8 months or more depending on the condition of the subject recipient and the donor transplant. In certain embodiments, anti-CD3 immunotoxin can be used to selectively control T cells while sparing effects on T-regulatory cells avoiding adverse effects observed when using immunosuppressant agents and the like. In accordance with these embodiments, by reducing the need for immunosuppressive agents, a subject recipient can avoid the side effects of these agents at a reduced cost with improved outcomes. In some embodiments, a subject recipient of a transplant can be treated with anti-CD3 immunotoxin for about 1 day, to about 2 days, to about 3 days, to about 4 days or about one week or about 2 weeks or more, depending on need and other factors such as the transplant type for example.

In other embodiments, compositions and methods are disclosed for modulating or depleting T cells in a subject scheduled for, undergoing or having undergone transplantation or implantation. In certain embodiments, a subject scheduled for, undergoing or having undergone a transplantation or implantation can be treated with a composition including, but not limited to, one or more anti-CD3 immunotoxin for reducing the risk of, preventing or treating transplantation rejection. In certain embodiments, compositions including, but not limited to, one or more anti-CD3 immunotoxin can be used to treat an acute T-cell mediated rejection episode in a subject having received a transplanted organ, tissue and/or cells to reduce or prevent rejection in the subject and improve transplantation outcome. In other embodiments, compositions including, but not limited to, one or more anti-CD3 immunotoxin can be combined with other agents to treat rejection including acute rejection. In other embodiments, compositions including, but not limited to, one or more anti-CD3 immunotoxin when administered to a transplant recipient can reduce the need for immunosuppressive agent treatment regimens, eliminate the need for immunosuppressive agent treatment regimens or modulate these regimens to reduce side effects of these treatments while reducing transplantation rejection in the subject.

In certain embodiments, the subject is at risk of developing or has graft versus host disease (GvHD). In accordance with these embodiments, a subject can be administered a composition including, but not limited to, one or more anti-CD3 immunotoxin to reduce the onset of, prevent or treat GvHD in the subject. In other embodiments, the subject has acute, chronic or steroid-refractory GvHD and compositions and methods disclosed herein treat the acute, chronic or steroid-refractory GvHD; for example, by modulating T-cells in the subject. In other embodiments, compositions including, but not limited to, one or more anti-CD3 immunotoxin when administered to a transplant recipient can reduce the need for immunosuppressive agent treatment regimens, eliminate the need for immunosuppressive agent treatment regimens or modulate these regimens to reduce side effects of these treatments and treat, reduce onset of or prevent GvHD.

In certain embodiments, one or more immunotoxin can be used to pre-treat a subject undergoing a transplantation event. In some embodiments, the one or more immunotoxin includes, one or more anti-CD3 immunotoxin can be generated using recombinant technologies. In some embodiments, the one or more anti-CD3 immunotoxin can be a fusion molecule. In other embodiments, the one or more anti-CD3 immunotoxin can be a fusion molecule for use in humans. In some embodiments, anti-CD3 immunotoxin can be generated specific for a subject such as a human or non-human subject. In certain embodiments, a recombinant fusion toxin includes, but is not limited to, an anti-human CD3 binding domain and a truncated diphtheria toxin. In certain embodiments, the truncated diphtheria toxin includes, but is not limited to, chain A. In other embodiments, an anti-CD3 immunotoxin can include an anti-human CD3 binding domain having an anti-human CD3 epsilon specific monoclonal antibody segment or full monoclonal and the truncated diphtheria toxin can include a translocation and catalytic domain of a truncated diphtheria toxin.

In certain embodiments, the anti-CD3 immunotoxin can be Resimmune® (A-dmDT390-bisFv(UCHT1)) or other anti-CD3 immunotoxin. In some embodiments, the anti-CD3 immunotoxin includes a bivalent anti-T cell immunotoxin, A-dmDT390-bisFv(UCHT1). The diphtheria toxin moiety has been modified to include an NH2 terminal alanine (A) and two double mutations (dm) have been made to prevent glycosylation in the eukaryotic expression system. The bivalent immunotoxin, A-dmDT390-bisFv(UCHT1) contains the first 390 amino acid residues of diphtheria toxin (DT) and two tandem sFv molecules derived from UCHT1 parental antibody. The first 390 amino acid residues of DT (DT390) contain the catalytic domain or A chain of DT that inhibits protein synthesis by ADP ribosylation of elongation factor 2 (EF-2) and the translocation domain that translocates the catalytic domain to the cytosol by interaction with cytosolic Hsp90 and thioredoxin reductase. A-dmDT390-bisFv(UCHT1) passes through the bloodstream and binds the CD3 positive leukemic cells in bloodstream and lymphatic system. In association with two helices of the translocation domain (TH1 and TH2: residues 201-230 of DT), cytosolic Hsp90 and thioredoxin reductase, the catalytic domain of A-dmDT390-bisFv(UCHT1) unfolds, is reduced, and translocates to the cytosol. The catalytic domain refolds and catalytically inactivates cellular protein synthesis by ADP-ribosylating the diphthamide residue in domain IV of EF-2. Other constructs and agents capable of binding and inhibiting development and/or expansion (e.g. transient) CD3 positive T-cells are contemplated herein. In some embodiments, the anti-CD3 immunotoxin can be designed to target a specific species (e.g. pig, dog, livestock, horse) in order to perform pre-clinical testing for example.

In some embodiments, anti-CD3 immunotoxin can be administered to the subject recipient several times per day, twice daily, daily, every other day or other regimen for targeting T cell suppression or kill T cells, such as selectively killing T cells. In one embodiment, the anti-CD3 immunotoxin can be used to kill normal within a day, a few days, a week or longer after treatment. In some embodiments, anti-CD3 immunotoxin of use herein provides advantages over other lymphocyte-depleting agents by having a short half-life allowing for rapid T cell recovery and improved immune competence, potent ability to rapidly deplete T cells within tissues as well as peripheral blood, while in some embodiments, relatively sparing T regulatory cells. Other agents, such as ATG® and Campath®, fail to deplete T cells in tissues and prolong immunosuppression for longer periods of time (for example, by prolonging peripheral blood T-cell depletion with adverse effects) in a subject receiving such an agent leading to disruption in normal regulatory responses and increasing risk of complications due to infection (e.g. bacterial, viral, fungal etc.). In some embodiments, a subject recipient of a donor organ, tissue or cells can be administered about 1.0 μg/kg to about 200.0 μg/kg (total body weight); or about 1.5 μg/kg to about 175.0 μg/kg (total body weight); or about 2.5 μg/kg to about 150.0 μg/kg (total body weight) or about 5.0 μg/kg to about 100.0 μg/kg (total body weight) or about 15.0 μg/kg to about 75.0 μg/kg (total body weight) or about 20 μg/kg or about 2.5 μg/kg of anti-CD3 immunotoxin more than one time daily, twice daily, one time daily, every other day or other appropriate regimen for a day, for about 4 days, for about 5 days, for about a week or more. In accordance with these embodiments, total treatment doses in a single course of treatment of a subject recipient can be about 1.0 μg/kg to about 200.0 μg/kg (total body weight); or about 5.0 μg/kg to about 175.0 μg/kg (total body weight); or about 7.5 μg/kg to about 150.0 μg/kg (total body weight) or about 10.0 μg/kg to about 100.0 μg/kg (total body weight) or about 15.0 μg/kg to about 75.0 μg/kg (total body weight) or about 20 μg/kg (total body weight). In some embodiments, if more than one dose is provided in a single day, each dose of anti-CD3 immunotoxin can be provided to the subject recipient about a minute, an hour, more than one hour, more than 2 hours, more than 4 hours, more than six hours, more than eight hours or more apart from one another or other regimen depending on status of the subject and need. In some embodiments, a subject recipient can be treated for about 5 to about 1 hour per infusion followed by a second or a third infusion or other appropriate schedule about 2 to about 8 hours apart for about 2 days to about 1 week. In accordance with these embodiments, a subject recipient can be treated with about 1.0-40 μg/kg in total for all doses. In some embodiments, a subject recipient can receive per dose administration about 0.1 μg/kg to about 20 μg/kg dose per administration or about 0.5-15 μg/kg per dose over several days (e.g. 2.5 μg/kg 2××4 Days or 1.25 μg/kg 2××4 Days or 0.625 μg/kg 2××4 Days) or a dose escalation starting dose, or a dose reducing dose for progression across the course of treatment. In some embodiments, the anti-CD3 immunotoxin can be infused into the subject recipient such as intravenous infusion or for example, free-flowing intravenous administration.

In certain embodiments, a combination composition including, but not limited to, one or more anti-CD3 immunotoxin and peripheral blood cells from the donor of the transplanted organ, tissue or cells are contemplated. In certain embodiments, the one or more anti-CD3 immunotoxin of the compositions can be a construct created by recombinant technologies. In other embodiments, the one or more anti-CD3 immunotoxin can be a fusion molecule and can further be a fusion molecule designed for the subject being treated (e.g. human subject). In certain embodiments, donor peripheral blood cells can include, but are not limited to, peripheral blood mononuclear cells (PBMC).

In certain embodiments, a combination composition including, but not limited to, one or more anti-CD3 immunotoxin and immune cells from the donor of the transplanted organ, tissue or cells are contemplated. Immune cells can be categorized as lymphocytes, neutrophils, granulocytes, mast cells, monocytes/macrophages, and dendritic cells. In some embodiments, combination compositions disclosed herein can include lymphocytes. In accordance with these embodiments, lymphocytes for use herein can be T-cells (CD4 T cells and/or CD8 T cells), B-cells, and natural killer (NK) cells are categorized as lymphocytes. In some embodiments, combination compositions disclosed herein can include at least one allogeneic immune cell. As used herein, the term “allogeneic” can refer to a peripheral blood, umbilical cord blood, bone marrow, PBMCs, leukapheresis sample, tumor-infiltrated lymphocytes, tissue-infiltrated lymphocytes, lymph nodes, thymus, and/or secondary lymphoid organs obtained from in this case, the donor of the organ, tissue or cells. In some embodiments, an immune cell can be isolated from haploidentical allogeneic peripheral blood, umbilical cord blood, bone marrow, PBMCs, leukapheresis sample, tumor-infiltrated lymphocytes, tissue-infiltrated lymphocytes, lymph nodes, thymus, and/or secondary lymphoid organs.

In certain embodiments, the subject is preparing for, undergoing or has had a solid organ transplant. In certain embodiments, the subject is preparing for, undergoing or has had a kidney transplant. In other embodiments, the donor kidney can be from an MHC fully matched, a partial MHC matched (e.g. haplo-mismatched) or a fully MHC mismatched donor compared to the subject receiving the transplant.

In certain embodiments, compositions and methods disclosed herein can be used for preparing a subject for organ, tissue and/or cellular transplantation. In some embodiments, compositions and methods disclosed herein can be used to reduce transplant rejection in the subject and prolong transplant survival. In other embodiments, a single treatment of one or more anti-CD3 immunotoxin and a single treatment of peripheral blood cells from the donor of the organ, tissue or cells can permit more than one transplantation event to occur of the same type or different types as long as the donated organ, tissue or cells are obtained from the same donor. Alternatively, as needed, peripheral blood cells can be obtained and provided to the subject recipient from each donor if more than one donor is providing an organ, tissue and/or cells. In certain embodiments, skin grafts or transplants can require more than one graft event in order to fully treat as subject; for example, a burn victim, an accident victim or a subject suffering from multiple organ failures. It is contemplated that the subject recipient receiving more than one graft can be tolerant without the need for subsequent anti-CD3 immunotoxin and/or donor peripheral blood cell treatments. In other embodiments, subsequent anti-CD3 immunotoxin and/or donor peripheral blood cell treatments can be provided to a subject having more than one graft event. In certain embodiments, cellular implantation can require more than one implantation event. It is contemplated that the subject recipient receiving more than one graft can be tolerant without the need for subsequent anti-CD3 immunotoxin and/or donor peripheral blood cell treatments. In other embodiments, subsequent anti-CD3 immunotoxin and/or donor peripheral blood cell treatments can be provided to a subject recipient having more than one graft event. In some embodiments, kits are contemplated. In accordance with these embodiments, a kit can include one or more anti-CD3 immunotoxin and peripheral blood cells from the donor; and at least one container. In other embodiments, kits can further include one or more delivery device. In yet other embodiments, kits can include devices for obtaining peripheral blood cells from the donor for collection and/or storage and further include one or more anti-CD3 immunotoxin.

In certain embodiments, disclosed herein are kits for assaying samples for one or more biomarkers contemplated herein for presence, absence or concentration levels of the biomarkers. Kits according to the present disclosure can include one or more reagents useful for practicing one or more immunoassays according to the present disclosure.

In certain embodiments, kits of use here can be used to assess presence of or level of donor specific antibodies in a subject treated or not treated by compositions and methods disclosed herein. In accordance with these embodiments, reagents and assays for measure donor specific antibodies is contemplated. In some embodiments, positive and negative controls can be included in the kits.

### EXAMPLES

The following examples are included to illustrate certain embodiments. It should be appreciated by those of skill in the art that the techniques disclosed in the examples which follow represent techniques discovered to function well in the practice of the claimed methods, compositions and apparatus. However, those of skill in the art should, in light of the present disclosure, appreciate that changes can be made in some embodiments which are disclosed and still obtain a like or similar result without departing from the spirit and scope of the invention.

### Example 1

In one exemplary method, experimental miniature swine (n=7) were used in a transplantation type model to analyze donor antibody production under selected conditions. In this example, donor antibody production was assessed in the test animal model after receiving ITC (a particular type of CD3 immunotoxin conditioning) conditioning together with infusion of various doses of cytokine mobilized or unmobilized peripheral blood mononuclear cells from haplo- or fully MHC-mismatched donors. Cytokine mobilized peripheral blood cells can include stem cells sufficient to engraft into recipient bone marrow following transplantation, while unmobilized peripheral blood cells typically do not include stem cells or can contain a few stem cells that are insufficient to support engraftment. Haplo MHC-mismatched donors were mismatched with recipients at one allele at both class I and class II loci, while fully MHC-mismatched donors were mismatched with recipients at both alleles at both class I and class II loci. These methods and protocols are sufficient to overcome any degree of MHC class I and class II mismatch, to provide for B cell tolerance (for example, B cell tolerance results despite the degree of MHC mismatch). Control miniature swine (n=8) received a) donor cell infusion without ITC conditioning (n=2), b) ITC conditioning without the infusion of donor cells (n=2), or c) neither ITC conditioning nor donor cell infusion (naïve controls, n=4). Experimental and control animals were injected with donor cells subcutaneously or challenged with a donor skin graft following cessation of CyA. In another experiment, anti-donor antibody responses were measured by for example, complement-dependent antibody-mediated cellular cytotoxicity assays and by flow cytometry to detect serum antibody mediated cytotoxicity/binding to donor versus recipient-matched target cells. Immunophenotyping of peripheral blood by flow cytometry was performed weekly in ITC conditioned animals to assess kinetics of depletion and recovery of leukocyte populations.

It was observed that experimental animals maintained undetectable donor specific antibody responses despite multiple challenges with donor cells and/or skin graft. In contrast, all control animals developed strong anti-donor antibody responses following donor cell or skin graft challenge.

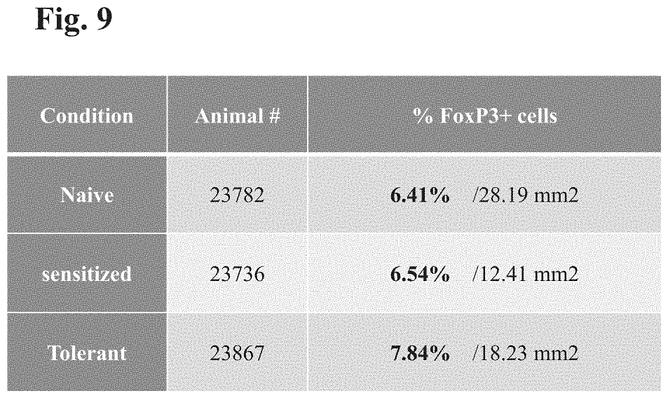

It was further observed that phenotypic analysis revealed that T cell depletion was rapid and transient with recovery of CD3+ T cells to levels observed in age-matched naïve controls within about 1-3 weeks. Among the CD3+CD4+ T cells remaining at the time of donor cell infusion on day 0, there was an increase in percentage of FoxP3+ cells, suggesting a relative sparing of T regulatory cells. See for example FIG. 9 Histological analysis of draining lymph nodes surgically removed 6 days following subcutaneous challenge with donor cells shows similar morphology of active B cell follicles in both groups. Preliminary analysis reveals an abundance of FoxP3+ cells observed within B cell follicles in experimental compared with control animals.

A reduced intensity conditioning (RIC) regimen for transplantation, using mobilized or unmobilized hemopoietic cells (HCT) has been described for use in MGH miniature swine (designed as an experimental model of use herein) and Rhesus macaques. RIC has reduced conditioning treatment that which are shorter treatment periods or reduced length of time, shorter than the current state of the art therapies for depleting T- and/or B-cells. Both the swine and non-human primates (NHP) are suitable preclinical animal models with established predictability for clinical translation. For example, the porcine immune system, despite some phenotypic differences, resembles that of humans more closely than rodents. Porcine immune responses resemble humans for 80% of analyzed parameters whereas mice are similar in less than 10%. The MGH miniature swine used are inbred at the MHC locus, enabling defined transplantation studies. In this preclinical model, stable stem cell engraftment was achieved without significant graft versus host disease (GvHD) when stem cells are mobilized with SCF and IL-3. When donor stem cells do not engraft, this protocol induces transient T-cell hyporesponsiveness to donor antigen but persistent humoral unresponsiveness of use in transplantation models and other health condition treatment where donor tissue is used.

In these experiments, the ability to induce humoral tolerance in full MHC mismatch, was demonstrated with stability over multiple donor antigen challenges. Surprisingly, mobilization of donor cells from the bone marrow and HCT was and is not required. Rather, donor specific immune hyporesponsiveness was induced with transfer of peripheral blood mononuclear cells (PBMC) (PBT), without mobilization. This RIC regimen induces less pronounced B- and T-cell depletion than conventional experimental and clinical conditioning regimens. Further, the recipient's immune system is preserved with reduced side effects than existing protocols. These experiments demonstrated humoral tolerance by either preserving pre-existing regulatory elements or by promoting antigen specific immune anergy but it does not appear to annul the germinal center reactions (GCR) in draining Lymph nodes. It was also observed that irradiation may be avoided. For example, because stem cell engraftment may not be necessary for the disclosed protocol, irradiation may be avoided.

In one example, a protocol used herein included administration of an immunosuppressant. The immunosuppressant was Cyclosporine A (CyA). Other immunosuppressants are contemplated, such as, for example Tacrolimus (e.g., for kidney transplantation in the clinic). In one example, the immunosuppressant was administered prior to PBMC infusion. For example, the immunosuppressant was administered prior to immunotoxin treatment and PBMC infusion. In some cases, the immunosuppression can be removed, for example, the recipient can be weaned from standard immunosuppression.

Chronic graft rejection in solid organ transplantation is thought to be primarily caused by AMR and remains a formidable challenge that has not been sufficiently addressed by existing therapies. Described herein are experiments showing that a strong humoral alloimmune response against haplo- and full-MHC mismatched donor cells may be greatly reduced and/or eliminated in animals treated with the present ITC protocol. In the present studies, humoral tolerance was shown to persist in the absence of both peripheral chimerism and donor cells engraftment in the recipient's lymphatic tissues. These results surprisingly show that: 1) B-cell tolerance may be achieved across a full MHC mismatch, 2) humoral tolerance may not be broken by repeated exposure to donor cells, 3) mobilization of stem cells from the donor bone marrow is not required, and 4) the GCR in the local lymph node is not completely prevented but unproductive.

In contrast to other approaches, the disclosed regimen does not aim for engraftment of donor stem cells in the bone marrow of the recipient. Consequently, peripheral donor chimerism was short-lived and no donor MHC could be detected in the myeloid and lymphoid tissues of experimental animals. Despite the lack of chimerism and engraftment, DRA were absent long after the transient period of peripheral chimerism. This humoral unresponsiveness was robust even when the animals were repeatedly challenged with subcutaneous. and intravenous exposure to donor cells or tissue. Skin grafts from donor animals did not evoke DRA formation either, despite vigorous skin graft rejection. The rejection of skin grafts was likely mediated by cellular alloimmunity, as a return of cellular donor specific immunity after a 7 to 14-week period of T-cell hyporesponsiveness. Despite the transient nature of cellular immune tolerance, humoral donor reactivity was not detectable throughout the animal's lifetime. For example, humoral donor reactivity (e.g., anti-donor antibody response) was not detectable for more than 240 days following transplantation (e.g., donor cell infusion with ITC conditioning). As another example, humoral donor reactivity was not detectable for at least 475 days following transplantation.

It is noted that humoral tolerance can be maintained by apoptosis and/or anergy of donor reactive B-cells, induced by peripheral regulatory mechanisms. Regulatory cells can be preferentially expanded during the conditioning period and exposure to donor PBMC. B-cell anergy can be induced by the absence of activating cytokine and cellular signals from T-helper cells and/or generated by education from donor T-cells. Other mechanisms can include indirect mediation by the cells and factors that control B-cell activation, such as TFH, TFR, B-regs, and follicular dendritic cells (fDC). It has been demonstrated that TFR cells limit the activating helper cell function of TFH and B-regs and CD8+T-regs are also capable of controlling the GCR.

While transient IgM class of DRA has occasionally been detected following challenges combined with complete Freund's adjuvant, it did not progress to the formation of DRA of IgG subclass after IgM DRA waned. This further suggests that the GCR is defective in leading to somatic hypermutation and class switching to pathogenic IgG DRA, reminiscent of incomplete extrafollicular B-cell responses. In addition, fDCs can induce and activate T-regs or delete reactive B-cells by presenting donor antigen. This mechanism has been described in the context of deleting self-reactive B-cells by fDC as a means of peripheral self-tolerance.

As such, the findings support that the GCR in response to a donor cell challenge is either defective and/or suppressed. Phenotypically, the draining lymph nodes of tolerant animals did not differ from naïve or reactive lymph nodes. Therefore, the process may be unproductive due to an active or passive dysfunction of affinity maturation or plasma cell differentiation. None of the markers of germinal center activity explored, were substantially different in unproductive, tolerant from productive, naïve or sensitized lymph nodes. The observed trend to a higher quantity of regulatory T-cells in Lymph nodes from tolerant animals may reflect an increase in immunoregulation. It is noteworthy that a scarce number of FoxP3+ was seen within Follicles and Germinal centers. The scarcity of these TFR cells and other regulatory cells such as B-regs, NK cells and MSDC67 in the germinal center may explain why differences in these cell populations were not detectable by bulk RNA analysis of lymph node cells.

The implications from the present results are of considerable clinical relevance. Patients with symptomatic AMR have significantly worse long-term outcome than stable patients or those that experience other forms of rejection, such as acute cellular rejection. Unlike cellular rejection, there are currently no effective treatment options for AMR. While non-immunological factors also impact graft survival, the majority of graft loss (especially for renal transplants) is associated with the presence of DSA directed against HLA and about half of patients have lost their graft 18 months after the diagnosis of chronic AMR has been made. The effective and permanent humoral tolerance induced with the disclosed RIC protocol may present a permanent therapy for AMR which has been elusive thus far. Existing therapies are limited to salvage therapies in acute AMR (complement targeted therapy) or are effective only prior to DSA formation (co-stimulation blockade). De-sensitizing therapies (B-cell depletion, anti-IL-6) are aggressive and of limited effectiveness. No other emerging therapy for AMR induces effective, active tolerance mechanisms. In addition, the present conditioning regimen is well tolerated and requires only a small amount of donor PBMC, rendering it a potentially attractive therapeutic avenue for AMR.

In addition, these findings improve the understanding of transplantation tolerance by suggesting that, contrary to widespread clinical use as induction therapy, thorough and enduring depletion of T- and B-cells (e.g., by ATG and Rituximab) may inadvertently destroy regulatory elements or prevent their formation. There is evidence that the initial allo- and autoimmune response is based in the activation of T-reg cells, a potentially crucial mechanism to induce operational transplant tolerance. Therefore, the clinical practice of immunosuppression as well as experimental tolerance induction therapies using T- and B-cell depletion should be reconsidered as it might interfere with humoral tolerance induction or maintenance.

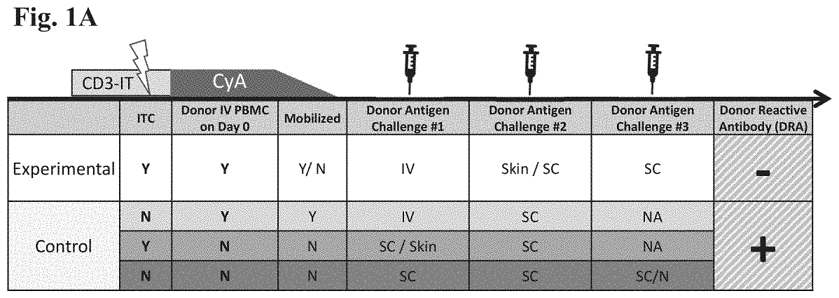

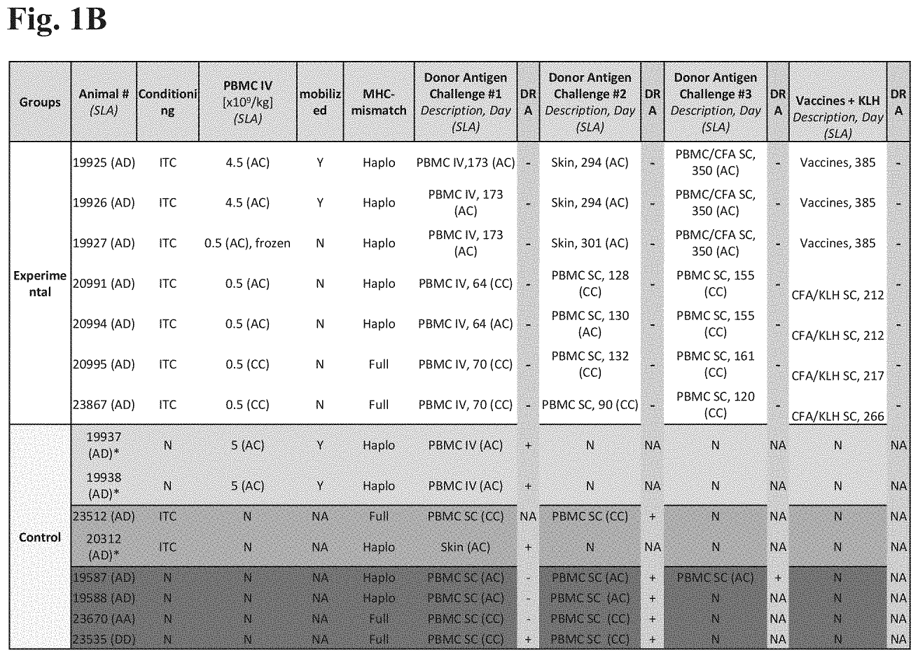

In one exemplary method experimental animals were used to assess methods for improving graft tolerance in the animals of use to evaluate and/or project use of these methods to improve transplant tolerance in human subjects. FIGS. 1A-1B represent schematics timelines and procedures of treated and untreated animals of various embodiments disclosed herein.

FIGS. 1A-1B represents an overview of experimental animals. Schematic timeline and group overview (1A) and detailed tabular overview (1B) for all animals in this study. Animals are shaded according to the conditions of ITC (as disclosed below to include total body irradiation and treatment with cyclosporine in addition to anti-CD3 immunotoxin treatment) and DO IV PBMC exposure. The experimental animals received haplo- or fully mismatched PBMC intravenously, skin graft and/or subcutaneous challenge with donor cells. No, NA=Not Applicable, (−)=No donor reactive antibody response detected, (+)=Donor reactive antibody response detected, *=included. Immune competence was assessed by immunization with commercial vaccines or Keyhole limpet hemocyanin (KLH) and complete Freud's adjuvant (CFA). Control animals were challenged with allogeneic PBMC intravenously or subcutaneously. Donor reactive Antibody was assayed by complement dependent cytotoxicity and antibody binding assays.

CyA (Cyclosporine A—45 days) [symbol] =Total body irradiation, 100 cGy for ITC treatments. Y=Yes, N=2012.

### Example 2

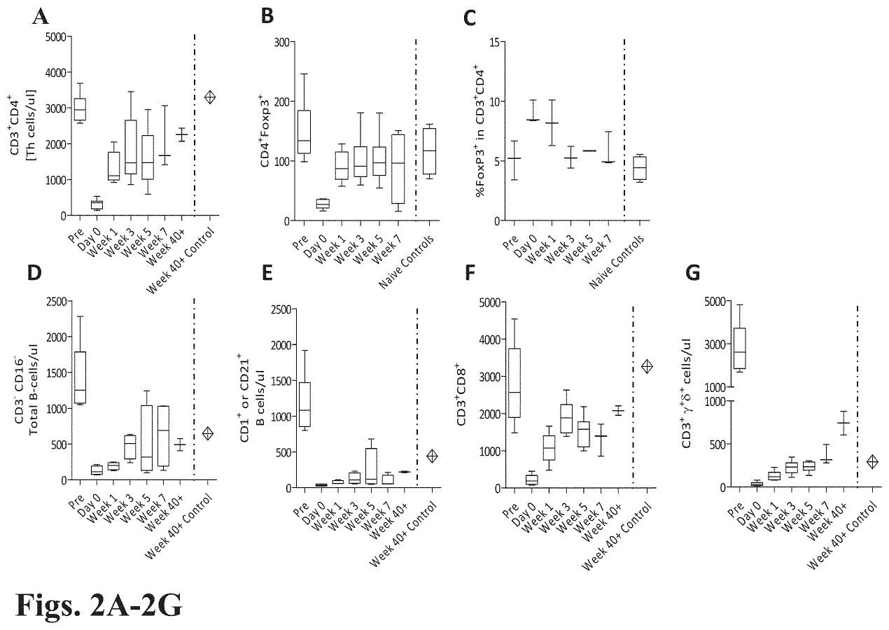

In one exemplary method, ITC conditioning and donor PBMC infusion results in subtotal T-cell depletion favoring T-regs and transient chimerism were analyzed. The CD3 immunotoxin given for 4 consecutive days, results in a significant but brief decrease of total CD3+CD4+ lymphocytes (3,000 to 321 CD3+CD4+ T cells/μl; FIG. 2A). T-helper cells recover quickly to near normal levels by 1-3 weeks (approx. 2,000 CD3+CD4+ T cells/μl; FIG. 2 A). The absolute quantity of CD4+FoxP3+ cells similarly recovers to near baseline levels within 1-3 weeks (FIG. 2B). However, the relative amount of regulatory T-cells (CD3+, CD25hi, FoxP3+) is increased by the ITC conditioning regimen from 5.1% up to 8.97% of total CD3+CD4+ T-cells during the first 2 weeks after conditioning until the proportion of FoxP3+CD4+ cells returns to baseline by week 3 (FIG. 2C).

CD3+CD8+ T-cells are also transiently reduced in the first week, following a similar recovery as CD3+CD4+T-helper cells (FIG. 2F). CD8+ T-cell population quantities from 3 weeks onwards until the endo of study, are within the lower range of baseline controls but below the levels of an age matched unconditioned animal. Neither CD4+, nor CD8+ T-cells were completely depleted with this regimen and recovery was considerably shorter compared to other T-cell depletion regimens (e.g. ATG). In contrast, CD3+ T-cells are profoundly depleted in the first week and do not reach baseline levels during the delayed recovery period. There was a second increase in T-cells quantities from week 7 until the end of study. This second increase suggests a minor expansion of the T-cells cell population in ITC treated animals relative to the age matched control animal (FIG. 2G).

Total B-cells, defined here as CD3−CD16−, are considerably reduced by the ITC conditioning and recover incompletely to about 45% of baseline values by weeks 5-7 (636K versus 1423K cells/μl, FIG. 2D). The subpopulations of CD1+ and CD21+ B-cells were particularly affected by ITC conditioning and nearly completely depleted, recovering to similar levels as demonstrated in age-matched controls (FIG. 2E).

FIGS. 2A-G represent exemplary immunophenotyping pre- and post ITC conditioning (2A-2C) T-helper cell populations before and at various timepoints after ITC conditioning, with (2A) illustrating all CD4+ T-cells, (B) CD4+FoxP3+T-regulatory cells and (2C) percentage of T-regulatory cells (CD4+FoxP3+) of all CD4+T-helper cells (2D-2E) B-cell populations, defined as CD3− and CD16−, (2F) CD3+CD8+ T-cells (2G) CD3+γλ T-cells. Experimental animals were an average of 12.2 weeks old on day 0 and naïve control animals were 16 weeks of age at the time of immunophenotyping. Control values matched in age to experimental animals at 40+ weeks are from a 1-year old naïve control animal of the same genotype.

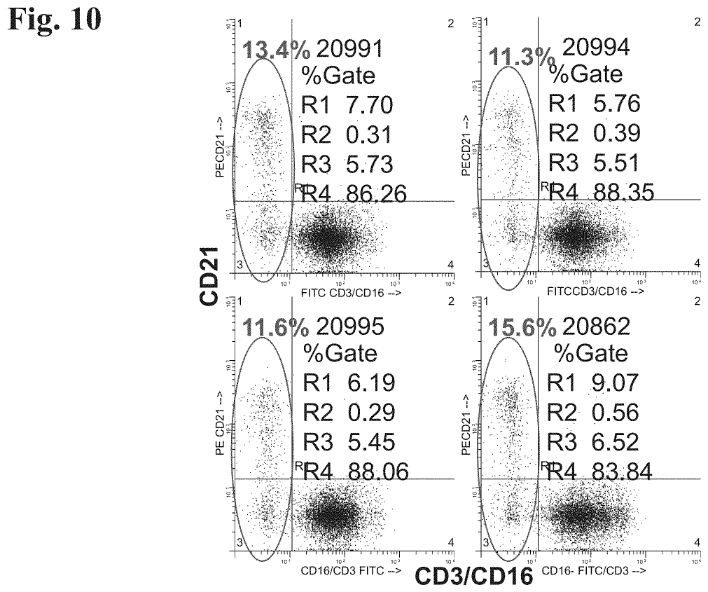

FIG. 10 illustrates flow cytometry experiments representing percentage of CD21+ and CD21-peripheral blood B cells (negative for CD3 and CD16 within the lymphocyte gate) is similar in “B cell tolerant” pigs after ITC conditioning compared to naïve control (20862).

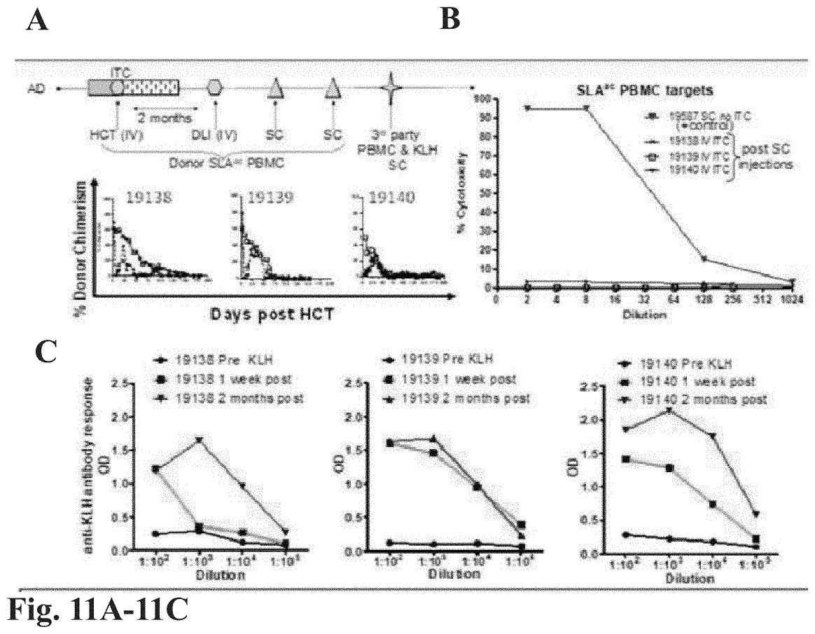

FIGS. 11A-11C are representative of ITC conditioned swine that lost chimerism and were repeatedly exposed to donor cells IV and SC prior to KLH immunization (11A). No cytotoxic anti-donor Ab was detected at any time-point (11B). Normal Ab responses were detected early (1 week) and persisted late (2 months) post KLH immunization (11C).

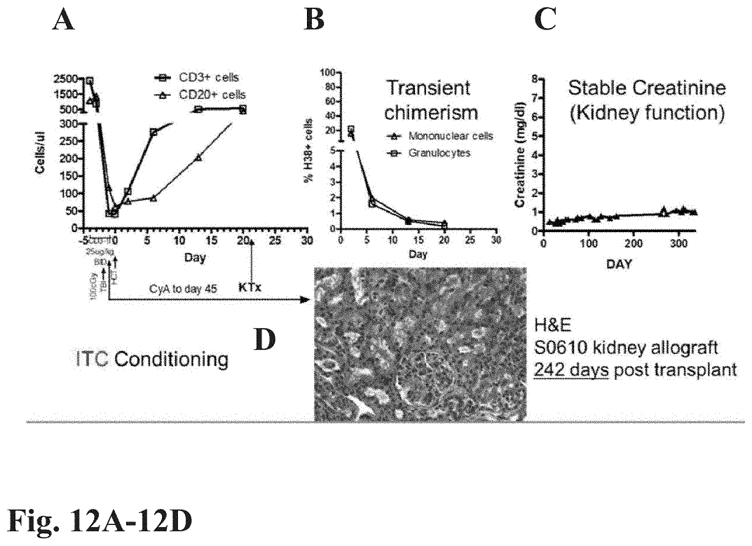

FIG. 12A-12D represent graphical illustrations of levels of various T cell populations, CD8+, CD4+ and T regulatory cells and the fluctuation of these cells after treatments using anti-CD3 immunotoxin. These data support that immunomodulation rather than immune ablation is responsible for the immune tolerance observed with this mild conditioning protocol. In a study in monkeys (FIG. 12A-12D), MHC-mismatched kidney allografts were accepted long-term after conditioning with similar cell-based induction protocol with CD3 immunotoxin. These results support the scientific premise that when a donor kidney allograft is placed during the early period when both donor specific T- and B-cell unresponsiveness is observed, stable T-cell tolerance is maintained in addition to the stable B-cell tolerance already achieved.

### Example 3

### Donor Chimerism is Transient and PBMC do not Engraft in Lymphoid and Myeloid Tissues

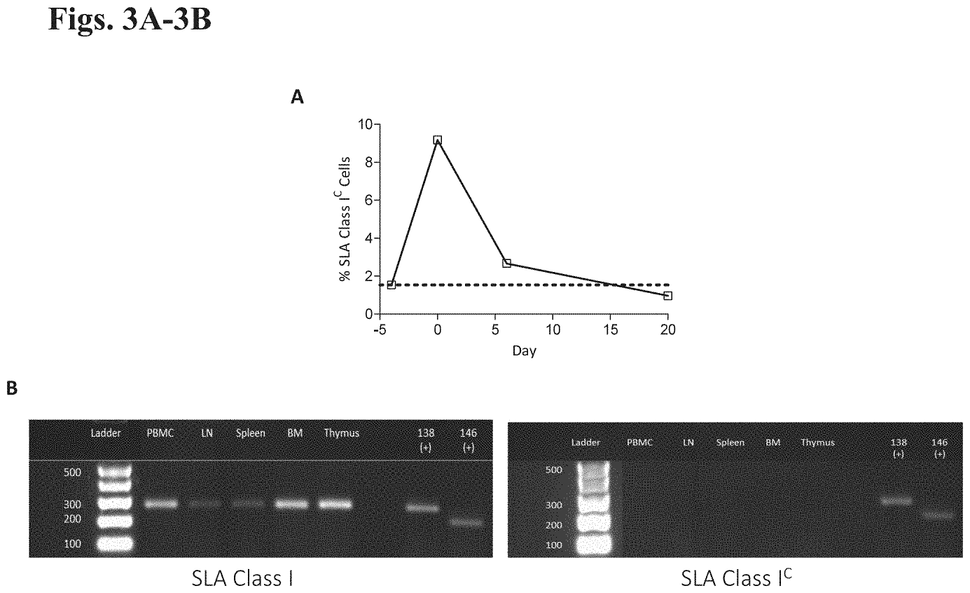

In another exemplary method, SLA class Ic positive donor cells were detected in the recipient's peripheral blood (9.2%) on the day of PBMC infusion and 6 days after (<3%; FIG. 3A). Twenty days following the first intravenous. infusion of donor PBMC, no more donor PBMC were detected in peripheral blood. At the time of sacrifice, samples from peripheral blood, Lymph nodes, Spleen, Bone marrow and Thymus were assessed for the presence of donor type SLA type Ic. The absence of donor MHC suggests that donor cells did not engraft and persist in the recipient (FIG. 3B).

This transient chimerism and failure to engraft was expected for ITC conditioned recipients of non-mobilized PBMC. Conversely, the majority of HCT recipients of mobilized donor PBMC exhibited persistent peripheral chimerism and stem cell engraftment. However, in 15% of recipients (9/60) that received mobilized donor PBMCs, the donor cells did not engraft. The present experiments were intended to investigate whether methods and protocols that avoid persistent chimerism and engraftment could allow for humoral tolerance.

FIGS. 3A-3B illustrate donor chimerism and engraftment (3A) represents donor peripheral blood chimerism returns to baseline within 3 weeks. Dotted line represents the background level of SLA class IC determined pre-treatment. (3B) represents no donor SLA class IC could be found in peripheral blood nor the lymphoid tissues spleen, thymus, bone marrow and lymph nodes at end of study. Representative data illustrated for a particular test animal.

### Example 4

### Naïve Pigs Exhibit Strong Humoral Responses to Donor Cell Challenges

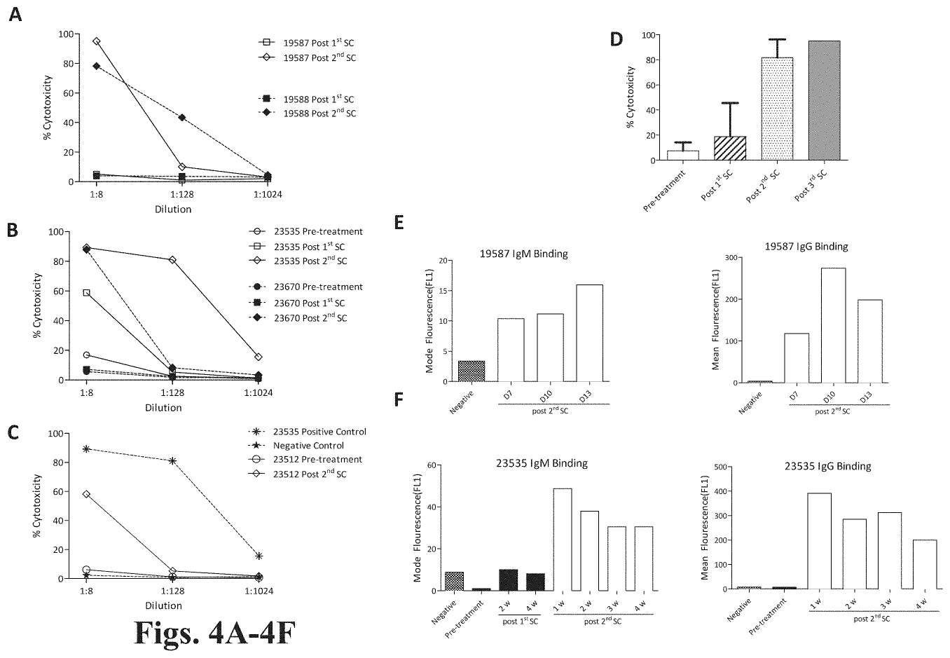

In another method, naïve MGH miniature swine that did not receive any prior conditioning and had never been exposed to cells or tissues of the donor or other pigs were challenged with donor PBMC by intravenous and subcutaneous injection. Donor cells that were haplo (FIG. 4 panel A) or fully (FIG. 4 panels B and C) MHC mismatched elicited strong, durable humoral responses in the recipient animals. Donor reactive antibodies were detectable following a single intravenous challenge with donor PBMC. Conversely, following the primary subcutaneous challenge, only animal 23535 developed a humoral response as measured by cytotoxicity but the other control animals did not. When comparing the average cytotoxicity values regardless of MHC mismatch, all control animals show normal humoral responsiveness following the secondary and tertiary (19587) subcutaneous donor cell challenge (FIG. 4 panel D). Antibody binding results demonstrate the persistence of IgM and IgG antibodies following second subcutaneous challenge in haplo- (FIG. 4 panel E) and full-mismatch control animals (FIG. 4 panel F)

FIGS. 4A-4F illustrated graphs and histogram plots including normal antibody response to subcutaneous challenges across haplo and full MHC mismatch (4A) represents a cytotoxicity assay with donor target cells demonstrates the appearance of donor reactive antibodies following the second and third, but not first subcutaneous challenge in haplo-mismatched animals AD/AC (4B) represents a graph of donor reactive antibodies that are detectable after first (23535) and second (23670) subcutaneous challenge in two full-mismatch control animals. FIG. 4C represents a conditioned control animal 23512 that demonstrates donor reactive antibody following a second subcutaneous challenge. FIG. 4D represents a histogram plot of mean cytotoxicity values for all control animals at 1:8 dilution. FIG. 4E represents IgM and IgG that are present in serum of representative animal 19587 following a second, but not the primary subcutaneous challenge. FIG. 4E represents in an experimental animal that IgM and IgG become detectable after a second subcutaneous challenge. All samples labeled “Post” were taken between 2-4 weeks after challenge for optimal antibody analysis. Negative samples for serum binding assay represent an average of target cells stained with secondary detection antibody, and target cells with secondary detection antibody with FBS.

### Example 5

### ITC Conditioning and HCT/PBT Induces Stable Humoral Tolerance to Donor Cells

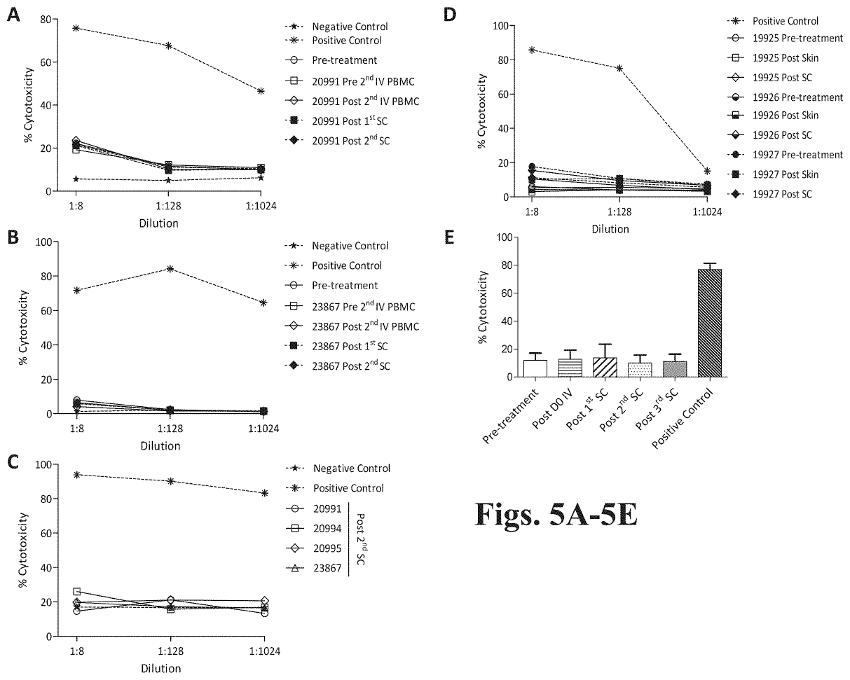

In one method, experimental animals receiving ITC conditioning with concurrent intravenous exposure to donor cells received multiple challenges with donor cells intravenous and subcutaneous across full- or haplo-MHC mismatches after cessation of all immunosuppression were analyzed. Unlike the control animals (FIG. 4), none of the experimental animals developed DRA at any time point following intravenous or subcutaneous challenges (FIG. 5). The serum of all animals was free of antibodies reacting with donor cells as assessed by cytotoxicity and antibody binding assays. There was no apparent difference between animals that received cells from mobilized or non-mobilized donor animals. In addition to humoral tolerance across haplo-mismatched donor recipient pairs, transfer of non-mobilized, fully mismatched donor PBMC rendered the recipient animals 20995 and 23867 tolerant (FIG. 5 panels B, C and E).

Donor PBMC were harvested by Leukapharesis with or without cytokine mobilization and administered via central venous lines as a HCT (4.5-5×10⁹ mobilized PBMC/kg) or donor PBMC infusion (0.5×10⁹ PBMC/kg) on day 0 and another donor cell infusion on day 70 (5×10⁷ PBMC/kg). Additional challenges were performed by subcutaneous injection of 80×10⁶ PBMC in the abdominal wall with drainage to the inguinal lymph nodes that were harvested for downstream analysis. Serum samples were analyzed at various time points preceding and following each challenge as outlined in FIG. 1 panel B. The serum did not contain any DRA at any of these time points following HCT, PBT, VCA graft and subcutaneous challenges (FIG. 3).

FIGS. 5A-5E represent a study illustrating the lack of antibody response following ITC/DO intravenously administered PBMC of some embodiments disclosed herein under multiple conditions The ITC conditioning regimen and DO intravenous PBMC exposure result in stable humoral tolerance towards the cell donor following multiple exposures across haplo and full MHC mismatches. FIG. 5A represents animal 20991 an AD recipient of AC donor HCT following ITC and receiving an intravenous donor cell challenge and two subcutaneous donor cell injections (SC). FIG. 5B represents animal 23867 and represents two animals receiving fully MHC mismatched donor cells by HCT, DLI and two SC challenges. FIG. 5C represents haplo and full mismatched animals did not make donor reactive antibodies after two intravenous and two subcutaneous donor cell challenges. FIG. 5D represents three animals that received a VCA graft following ITC, HCT and DLI which was subject to cellular rejection, however animals did not produce donor reactive antibodies even after an additional subcutaneous challenge. FIG. 5E is a display of mean cytotoxicity values for all experimental animals at a 1:8 dilution.

FIGS. 12A-12D illustrate long term acceptance of monkey kidney grafts despite loss of chimerism using a protocol involving CD3 immunotoxin and donor cell infusion where 12A-12C illustrate graphical representations of various immune cells, transient chimerism and a kidney function parameter while 12D represents a histological section of a kidney well after transplantation under conditions disclosed herein, anti-CD3 immunotoxin and donor cell infusion.

### Example 6

### Lymph Node Characteristics Following Subcutaneous PBMC Challenges

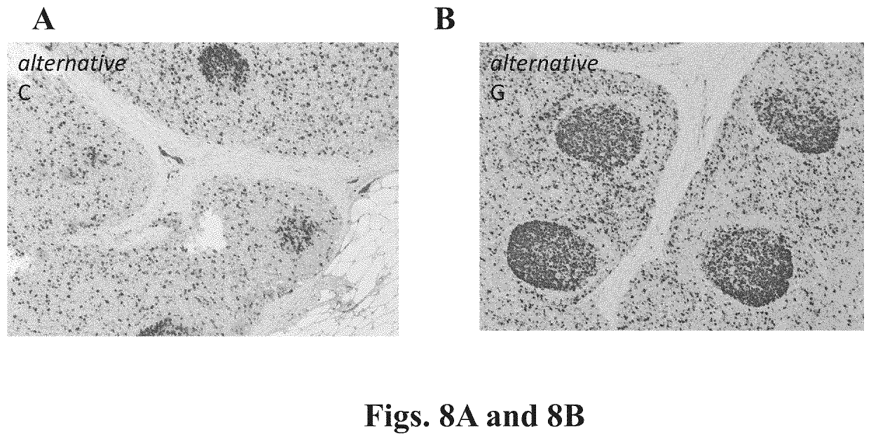

In another method, histologic analysis of lymph nodes draining the subcutaneous area where donor PBMC were injected demonstrates normal architecture including regular distribution of follicles, germinal centers and cells in the LN sinus. The quantity and distribution of CD20+ B-cells, PNA and GL-7+ intrafollicular B-cells, and CD21+ B-cells was comparable in lymph nodes from animals of the different groups. There was a slight increase in FoxP3+ cells in tolerant animal 23867 as compared to sensitized and naïve controls (see FIG. 8). Surprisingly, naïve lymph nodes were not significantly less mitotically active than activated sensitized or tolerant animals.

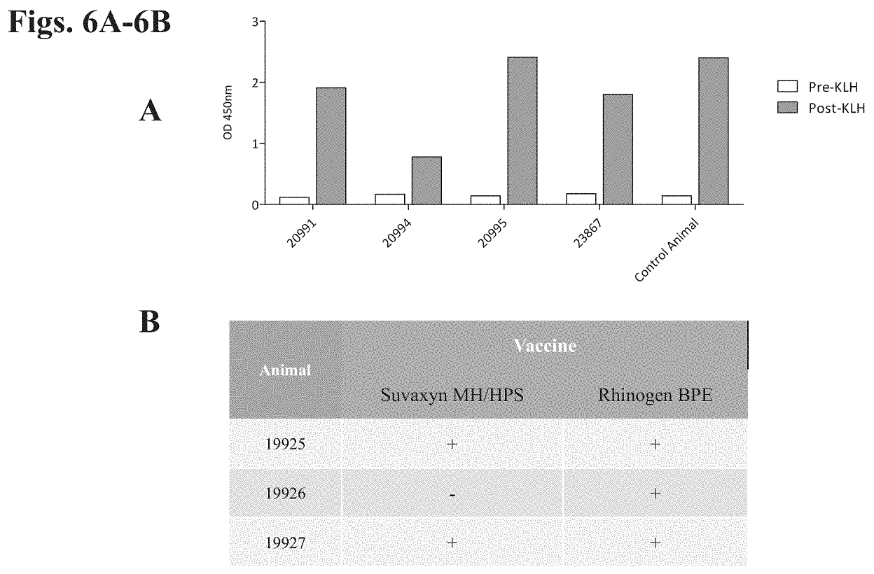

Overall, the present results suggest that tolerant animals have a phenotypically normal GCR, despite the absence of donor reactive IgG Antibodies (see for example, FIG. 6). In some cases, there may be differences in cellular composition or activity and interactions of follicular cells such as T follicular helper (TFH) cells, T follicular regulatory (TFR) cells, follicular dendritic cells (FDC), natural killer (NK)-cells and various B-cell subtypes. Transcriptional analysis was performed by bulk RNA sequencing, but differences between groups were not identified. In some cases, transcriptional profiles on a single cell level or of sorted cell populations may detect differences in the functional state of specific and/or rare cell types.

In another example, FIG. 6A-6B represents immune competence in animals unresponsive to donor challenges. In FIGS. 6A and 6B, animals that had no detectable antibodies against donor cells following three donor cell challenges were injected subcutaneously with keyhole limpet hemoacyanin (KLH) and complete Freud's adjuvant (CFA). Sample dilutions shown here are 1:1000. Positive control is shown in (6A). The data for animals 20991, 20994 and 20995 illustrated here is representative for all animals that were rendered tolerant. All tolerant animals retained the ability to generate antibodies against the immunogen KLH, similar to positive controls representative of a naïve animal but did not generate anti-donor antibodies. KLH antibody titers were measured by sandwich ELISA. In FIG. 6 B, as illustrated in exemplary FIG. 4, animals were tested for their humoral response to immunization with KLH, Mycoplasma Hyopneumoniae Bacterin (MH—Suvaxyn MH/HPS™) and Erysipelothrix rhusiopathiae (EP—Rhinogen BPE™). +=Antibodies detected; −=animal not immunized.

### Example 7

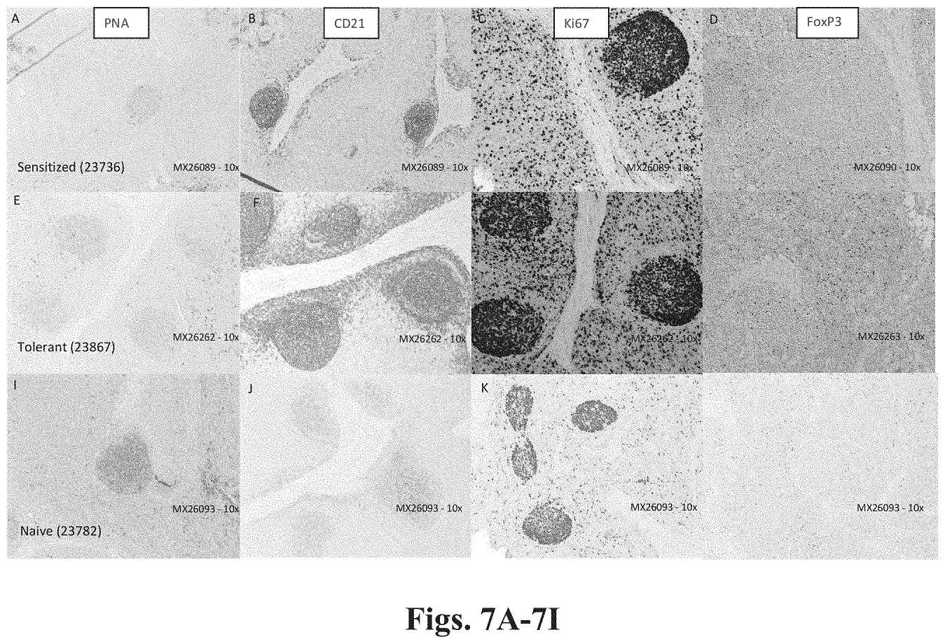

FIGS. 7A-7I represent histology of draining lymph nodes of various experimental animals. In these examples, the animals were subcutaneously injected with donor cells and draining inguinal lymph nodes were surgically removed 6 days later. Immunohistochemistry was performed with the antibodies PNA, CD21, Ki67 and FoxP3. (Row1) 23736 was pre-sensitized by subcutaneous donor cells 2 weeks prior to ITC (anti-CD3 immunotoxin) conditioning and then re-exposed to 70 million donor cells subcutaneous on day 61. Inguinal LN were removed on day 67. (Row2) Animal 23867 underwent the ITC/DO PBMC IV protocol and was re-exposed subcutaneous to donor cells on day 120 post ITC, LN were removed on day 126 (Row 3) Animal 23782 was an untreated, naïve donor animal whose naïve, unchallenged LN was removed surgically at autopsy. FIGS. 8A and 8B represent enlarged views of 7C and 7G above.

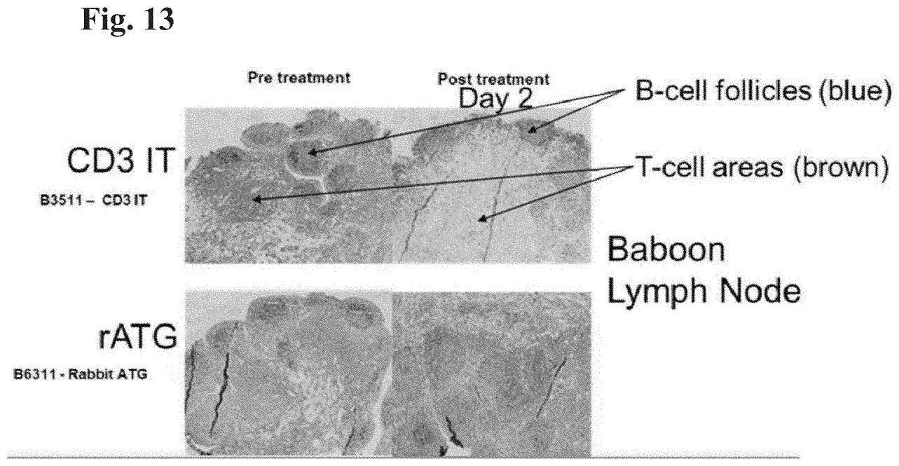

In these exemplary methods, the CD3 immunotoxin, Resimmune®, has distinctly different properties from other lymphocyte depleting agents currently being used in transplantation induction therapy such as Thymoglobulin-Genzyme, rATG-Fresenius, and Alemtuzumab (Campath). These properties include a short half-life, which allows rapid T cell recovery and improved immune competence; a potent ability to rapidly deplete T cells within tissues as well as in the peripheral blood; and a relative sparing of T regulatory cells. Therapeutic antibodies such as rATG and Campath are ineffective at depleting T cell within tissues (FIG. 13) and the prolonged half-lives of these reagents contribute to delayed immune reconstitution in patients, disrupting regulatory mechanisms, increased risk of autoimmune disease development and increasing the likelihood of infectious complications. It is thought that the unique properties of CD3 immunotoxin contributes to an immune regulatory response of the infused donor cells resulting in B-cell tolerance induction.

FIG. 13 illustrates a comparison of anti-thymocyte globulin (rATG) and CD3-immunotoxin (CD3 IT) analysis before and after treatment. The images represent pre-treatment (left panels) and post-treatment (right panels) of lymph node cross sections stained for the presence or absence of B-cell follicles (blue) and T-cell areas (brown) (colored image available upon request).

FIG. 9 illustrates a table comparing samples obtained from test animals regarding percentage of FoxP3+ cells in control versus experimental animals (tolerant) after treatment using compositions and methods disclosed herein.

### Example 8

### Human Clinical Trial

In another exemplary method, human subject needing a transplantation can be treated as disclosed and contemplated herein. Resimmune® is a novel bivalent T cell immunotoxin which incorporates diphtheria toxin (DT) and two tandem sFv molecules derived from the UCHT1 parental antibody (an anti-CD3 antibody). The first 390 amino acid residues of the Resimmune recombinant protein contain the catalytic domain or A chain of DT that inhibits protein synthesis by ADP ribosylation of elongation factor 2 (EF-2) as well as the translocation domain that translocates the catalytic domain to the cytosol by interaction with cytosolic Hsp90 (Heat Shock Protein 90) and thioredoxin reductase.

Objectives of this example are to determine the safety of A-dmDT390-bisFv(UCHT1) at a total dose of about 5 to about 20 μg/kg (total body weight) when administered to a human subject, determine a human subject's response to a treatment regimen of A-dmDT390-bisFv(UCHT1), and explore if T cell activation occurs following administration of A-dmDT390-bisFv(UCHT1).

### Preclinical Information on Resimmune®, A-dmDT390-bisFv(UCHT1)

Constructions. Plasmid encoding A-dmDT390-bisFv(UCHT1) molecule was made and integrated into Pichia pastoris genome, and recombinant proteins were produced in P. pastoris via the secretory route.

Selective cytotoxicity to cell lines. Inhibition of protein synthesis was measured after A-dmDT390-bisFv(UCHT1) incubation with Jurkat cell line and Vero cell line. Jurkat cells express CD3 on their cell surface, but Vero cells do not express CD3. Jurkat cell line was sensitive with IC₅₀ of 0.017 pM. Vero cell line was not sensitive at <10 pM.

Safety and efficacy in rodent models. Ten week old Balb/c female mice tolerated intraperitoneal infusions of A-dmDT390-bisFv(UCHT1) at a total dose of 500 μg/kg. All mice of the highest dose groups (1250 μg/kg in total) were dead due to severe acute tubular necrosis of kidneys. Twelve Sprague Dawley rats with femoral catheters received up to eight injections twice a day for 4 consecutive days through intravenous infusion of A-dmDT390-bisFv(UCHT1) at a total dose of 20 μg/kg (n=4), 200 μg/kg (n=4), or 450 μg/kg (n=4). Rats of the high dose group (450 μg/kg) survived and recovered after more than 20% weight loss. Except for AST levels, blood chemistry and CBC parameters were not significantly changed by drug administration. AST level in the high dose was 3.5 fold higher than that of the control group. AST level in the medium dose (200 μg/kg) was increased by 2 fold. The low dose group (20 μg/kg) was very similar to the control group.

Safety in monkeys. Nine squirrel monkeys received up to eight injections twice a day for 4 consecutive days, intravenous infusions of A-dmDT390-bisFv(UCHT1) at a total dose of 20 μg/kg (n=3), 200 μg/kg (n=3), or 450 μg/kg (n=3). After 24 days, all monkeys were necropsied. The MTD was 200 μg/kg and dose-limiting toxicity was reversible elevation of liver transaminases. Neither liver nor renal function was negatively impacted. No histopathological changes were observed. The drug half-life was 17.8 minutes, and immune responses were minimal. No significant myelosuppression or liver injury was seen.

### Clinical Treatment Schedule

Starting on day 1, 1 hour prior to each of the eight infusions of study drug, the patients receives premedication as needed. Vital signs are obtained at pre-treatment including temperature, respirations, Blood pressure (BP), pulse, and pulse oximetry. Starting on day 1 and repeated on days 2-4 after pre-medication and pre-treatment lab draws and vital signs measurements, A-dmDT390-bisFv(UCHT1) fusion protein is administered into a free flowing IV over a period of approximately 15 minutes. The second dose on day 1 and the doses on day 2, 3, and 4 are given in the absence of a drug toxicity response.

On day 1, vital signs are measured every fifteen minutes (15-minute post infusion+3 min, 30-minute post±5 min, 45 minute post±5 min, 1 hour post±10 min) for one hour and then hourly ×5 (2 hour post, 3 hour post, 4 hour post, 5 hour post, and 6 hour post infusion all±10 minutes). Vital signs are measured on days 2-4 every hour for 3 hours post infusions. Vital signs include BP, pulse, temperature, respirations and pulse oximetry. Daily weights are obtained to monitor fluid balance. Patients are required to have a temperature <100.5° F., Pulse <120 and >50, and Systolic BP<160 and >80 mmHg prior to therapy. Fasting blood sugars are checked during treatment days (days 1 through day 4) to see if insulin is required to treat hydrocortisone-induced hyperglycemia. EKG is obtained on days 1 immediately after completion of the first infusion and on day 4 immediately after completion of the eighth infusion. Patients are monitored until day 14 for signs of late drug toxicity by a daily phone call from a health care provider. Patients are instructed on how to monitor their own blood pressure at home and encouraged to measure and chart their daily weights that they can report to the inquiring health care provider.

Peripheral blood counts are monitored daily on days 1-5. Serum chemistries including albumin, alkanine phosphatase, BUN, calcium, creatinine, glucose, inorganic phosphorus, Lactate dehydrogenase (LDH), magnesium, AST, CPK, total protein, uric acid, bilirubin and DIC screen are done daily on days 1-5. Elevated prothrombin times (INR>1.3× normal) in the absence of other abnormalities consistent with DIC will be treated with vitamin K 5 mg IV daily for 4 days and, after the end of fusion protein infusions (day 5) with fresh frozen plasma as clinically indicated. Plasma should be avoided prior to completion of the four A-dmDT390-bisFv(UCHT1) infusions unless clinically necessary due to the likely presence of anti-DT antibodies in the blood product. Thrombopenia and anemia are treated with irradiated blood products as clinically indicated until completion of the A-dmDT390-bisFv(UCHT1) infusions again to avoid anti-DT antibodies in the blood products. Cryoprecipitate is used to replace fibrinogen for levels <100 mg/dL as cryoprecipitate does not contain significant anti-DT antibodies. Elevation of transaminases, grade 3 or grade 4, of less than 7 days duration require no treatment. Patients are officially withdrawn if they show obvious evidence of progressive disease while on therapy—prior to day-35 blood analysis. However, the administering clinician takes into account that the study drug has beneficial immunomodulatory effects that occur over a time span involving many months and mixed responses may occur.

Drug toxicity is graded according to the revised CTCAE version 5.0. Drug-related DLT is defined for this example as any non-hematologic toxicity of grade 3 or greater except for transient (<7 days) grade 3 or grade 4 asymptomatic elevations of transaminases or CPK and transient grade 3 and 4 lymphopenias lasting less than 28 days. Lymphopenia is not considered a DLT since it is the pharmacologic property of the study drug. Grade 3 reactivation of EBV and CMV are not considered DLTs since they are often associated with lymphopenia. EBV and CMV reactivations higher than grade 3 are considered DLTs.

### Drug Formulation, Availability and Preparation

A-dmDT390-bisFv(UCHT1) protein is an experimental drug with Approval of the FDA for investigational purposes. A-dmDT390-bisFv(UCHT1) protein is supplied frozen at 0.4 mg/mL in 5% aqueous glycerol solution with 0.15 M NaCl, 5 mM pH 8.0 Tris HCl buffer and 1 mM EDTA in 1 mL vials. New drug product vials can be used for each patient dose. Vials are thawed in a room temperature water bath, filter sterilized through a 0.2 μm filter (PALL Gelman Laboratory Acrodisc Syringe Filter 0.2 μm HT Tuffryn Membrane Low Protein Binding Non-Pyrogenic Ref: 4192 sterile), drawn into 1 mL calibrated tuberculin syringe and delivered into a 10 mL receiving syringe and diluted to 5 mL with sterile normal saline (USP) aseptically, and administered within 4 hours as a 15-minute infusion. The drug is to be given intravenously via a 5 mL plastic syringe or an infusion pump as an infusion of approximately 15 minutes on days 1-4 twice daily 4-6 hours apart. Directly following the drug infusion a second syringe with 2 mL normal saline flush will be administered.

### Materials and Methods

Resimmune® (anti-CD3 immunotoxin) is a strong, specific T cell depleting agent. Other anti-CD3 immunotoxins are contemplated of use herein and further can be adapted to the species to be treated such as human or non-human mammal (e.g. livestock, pets, horses).

Peripheral blood mononuclear cells (PBMCs) were prepared by various methods. In one example, the PBMCs were prepared by leukapheresis, where the population of PBMCs is between about 5×10⁷ CD3+ donor cells per kg of recipient body weight. In some examples, the population of PBMCs used was between 0.5-15×10⁹ donor cells per kg of recipient body weight. PBMCs can be less than about 0.5×10⁹ and more than about—15×10⁹ donor cells per kg of recipient body weight. PBMC population can be 0.5 billion donor cells per kg of recipient body weight or lower, for example, between 0.5×10⁵ donor cells per kg of recipient body weight to 0.5 billion donor cells per kg of recipient body weight.

### Animals

Animals were selected from a herd of partially inbred, major histocompatibility complex (MHC) defined miniature swine, which have been selectively bred over the past 40 years for large animal studies of transplantation. Through several generations of selective breeding, these swine have been defined at the MHC genes encoding for class I and class II antigens. This has allowed for reproducible transplantation studies across defined major histocompatibility antigen haplotypes while preserving minor histocompatibility antigen differences. Donors ranged from 4 to 23 months and recipients from 11 to 16 weeks of age at the time of HCT/PBT. Donors and recipients were chosen to differ by a single or both MHC haplotypes at both MHC-I and MHC-II, mimicking the clinical scenario of a related, haploidentical or fully mismatched transplant. To facilitate the detection of chimerism after HCT/PBT, only donors who were positive for pig allelic antigen (PAA) were selected. PAA is a non-histocompatibility cell-surface antigen that is present on all differentiated hematopoietic cells in animals that express this gene allele. All recipient animals were PAA-negative, so that donor cells could be detected by flow cytometry.

### ITC Conditioning

The ITC protocol disclosed herein is a combination of around 100 cGy total body irradiation, T cell depletion, and Cyclosporine A (CyA, about 45 days). It is noted that total body irradiation may not be needed if engraftment is not required. First, the animals received central venous lines that were inserted under general anesthesia 5-7 days before transplant. To this end, the internal or external jugular vein was identified and cannulated, the line was secured in place and externalized by creating a subcutaneous tunnel towards the retro aural region where the lines were attached to the skin superficially for secure access. A gastrostomy tube was placed at the same time as the central lines by midline laparotomy using the open Stamm procedure. A fogarty catheter was used and externalized via a subcutaneous tunnel towards the lateral side of the body. An oral microemulsion formulation of Cyclosporin A was administered via the gastrostomy or orally (p.o.). The dose was adjusted according to serum levels (target 400-800 ng/ml for the first 30 days, and tapered for the last 15 days to a level of 200 ng/mL, at which point CyA was discontinued). It was observed that these treatments with the immunotoxin were also capable of depleting T cells out of tissues supporting use of the immunotoxin therapies alone or in combination treatments.

Partial and transient host/recipient T cell depletion was achieved over 4 days using eight BID (e.g., two daily) doses of a recombinant CD3-immunotoxin starting 4 days before donor cell infusion (for example, Day −4). The drug was given via the central line following a dose of Diphenhydramine (2 mg/kg intravenous). Lastly, on day −2, recipient animals were subjected to 100 cGy Total body irradiation from a Cobalt source under general anesthesia.

### Mobilization, Leukapharesis, and PBMC Infusion

Peripheral blood mononuclear cells (PBMC) were harvested from donor animals by leukapheresis using a COBE Spectra apheresis system (AutoWBC set 777006-000; Terumo BCT), beginning on day 0 and repeated until the target cell number was attained. After the initial leukapheresis, 1 to 15×109 PBMCs/kg were infused intravenously daily until the target dose for HCT/PBT was achieved (day +1 in all cases).

Animals 19925, 19926, 19937 and 19938 received 4.5-5×10⁹ PBMCs/kg cells from donors that were mobilized for 5-7 days with porcine cytokines IL-3 and stem cell factor (SCF) (0.1 mg/kg for the first 30 kg of body weight and 0.05 mg/kg for each additional kg as described previously). Leukapharesis was performed beginning on day 5 of mobilization until the target amount was attained. All other conditioned animals in the experimental group received 0.5×10⁹ PBMCs/kg, without prior mobilization, for the first donor cell infusion.

A second, intravenous infusion of PBMC was performed 60 days after the initial donor cell exposure, following the completion of CyA treatment. The PBMC were harvested from fresh peripheral blood from the same donor animal and by Ficoll Gradient Separation (Ficoll-Paque PLUS, GE life sciences, Pittsburgh, Pa.). The cell dose was adjusted to contain 5×107 CD3+ donor T-cells per kg body weight as determined by Flow cytometry and repeated cell counts. Donor PBMC were administered in this example, via the central venous catheter or if central access was not available, via peripheral Angiocath.

### Subcutaneous Donor Cell Challenge and Lymph Node Biopsies

Freshly isolated or thawed PBMC from the original donor animal or from an animal with the donor MHC type were counted and diluted to 1.5 ml suspension of 1×108 PBMC. The animals were briefly anesthetized and the cell suspension was injected subcutaneously in the lower left (LLQ) or lower right quadrant (LRQ) of the abdomen, within the draining area of the respective inguinal lymph nodes. The injections were done either alone or in conjunction with complete Freud's adjuvant (CFA) and Keyhole limpet hemocyanin (KLH) as indicated.

After 6 days, the animals underwent surgical biopsy of the draining, inguinal lymph nodes from the ipsilateral side and non-draining lymph nodes from the contralateral groin. Prior to surgery, the cell injection site was infiltrated with Methylene blue to trace the lymphatic drainage of the area. Under surgical plane anesthesia, a 1-2 cm incision in the groin was made and 1-2 lymph nodes from the ipsi- and contralateral side were removed and processed for histology and RNA extraction. On the ipsilateral side, draining lymph nodes were identified by uptake or proximity to methylene blue labeled lymphatic vessels.

### Immunophenotyping and Assessment of Chimerism

Recipient animals were monitored during and following the ITC protocol for quantitative changes in peripheral immune cell composition. Using a multicolor flow cytometric panel cells were stained with pig specific antibodies CD3e (898H2-6-15; mouse IgGaK), CD4 (74-12-4; mouse IgG2bK), CD8a (76-2-11; mouse IgG2aK), CD172 (74-22-15; mouse IgG1K), CD5, and PAA (1038H-10-9; IgMK), CD16 (G7), CD1 (76-7-4), CD21 (BB6-11C9), FoxP3 (FJK-16s).

PAA is expressed on hematopoietic cells from PAA1 donor animals but not in PAA2 recipients. Peripheral blood chimerism was assessed by flow cytometry (FACS Calibur; BD Biosciences, San Jose, Calif.), as described previously. Autopsy samples of the animal's spleen, thymus and bone marrow were assessed for the presence of donor type MHC class Ic by PCR.

### Antibody Mediated Cytotoxicity Assay

Presence of donor specific cytotoxic antibodies were detected by complement-mediated cytotoxic assays. In brief, target cells were diluted to 5×106 cells/mL and suspended in Medium199 (Cellgro, Herndon, Va.) supplemented with 2% FCS. In 96-well U-bottom plates (Costar, Cambridge, Mass.), 25 mL of the appropriate target cell suspension was incubated with 25 mL of serum serially diluted from 1:2 to 1:1024 or controls for 15 minutes at 37° C., followed by a second incubation with 25 mL of appropriately diluted rabbit complement. Dead cells were identified by staining with 10 mL of 7-AAD for 30 minutes. Data were acquired, and the percentage of dead cells was assessed using a FACS Calibur (BD Biosciences) and analyzed with FlowJo software (Ashland, Oreg.).

### Antibody Binding Assay

A direct binding assay to detect the presence of donor reactive antibodies was performed by co-culturing donor cells with recipient serum. Following multiple washes, cells were stained with anti-porcine IgG1 (clone K139 3C8) and anti IgM (clone K52 1C3). Antibody bound to recipient cells was visualized with conjugated secondary antibodies and quantified by Flow cytometry.

### Immunohistochemistry

Tissue samples were fixed in 10% Paraformaldehyde and embedded in Paraffin. Sections of 5 μm thickness were incubated with antibodies against CD20, CD21, FoxP3, Peanut Agglutinin, GL7 and Ki67 and antibodies were visualized using DAB staining. Slides were reviewed with a Nikon Microscope and pictures were taken with Software. For quantification of positive cells, slides were scanned with an Aperio Slide scanner and analyzed with Aperio software (Leica, Buffalo Grove, Ill.). Normal germinal center reactions were observed in animals that did not make anti-donor antibody following donor cell challenge.

### RNA Sequencing

Lymph node tissue was harvested surgically as described above and a small tissue cube of approximately 2 mm2 was processed for RNA isolation using the RNAeasy Kit, including DNAse cleanup (Qiagen). Additional tissue samples were flash frozen. The isolated RNA was quantified using a NanoDrop (Thermo Fisher) and checked for integrity and sample quality using a Bioanalyzer (Agilent technologies). Samples that had a RIN score of 8 were processed for Library preparation using the Illumina stranded mRNAseq kit (Ilumina, San Diego, Calif.). The quality of the library was assessed using the Bioanalyzer (Agilent) and processed for sequencing on the NextSeq 550 Platform (Ilumina). Data analysis was performed with R Studio and graphs were plotted with Ggplot2 software.

All of the features disclosed in this specification may be combined in any combination. Each feature disclosed in this specification may be replaced by an alternative feature serving the same, equivalent, or similar purpose. Therefore, unless expressly stated otherwise, each feature disclosed is only an example of a generic series of equivalent or similar features.

From the above description, one of skill in the relevant art can easily ascertain the essential characteristics of the instant inventions, and without departing from the spirit and scope thereof, can make various changes and modifications of embodiments of the inventions to adapt it to various usages and conditions. Therefore, other embodiments are also within the disclosure as considered herein.

What is claimed is:

1. A method of inducing allograft tolerance in a recipient subject comprising:
administering prior to donor organ or tissue transplantation in the subject, a pharmaceutical composition comprising one or more anti-CD3 immunotoxin and a pharmaceutical composition comprising peripheral blood cells obtained from the donor of the donor organ or tissue and inducing allograft tolerance in the recipient subject to the donor organ.

2. The method according to claim 1, wherein administering the pharmaceutical composition comprising peripheral blood cells obtained from the donor comprises infusing the peripheral blood cell composition into the subject.

3. The method according to claim 1, wherein administering the pharmaceutical composition comprising peripheral blood cells obtained from the donor comprises administering the peripheral blood cell composition before, during, simultaneously, and/or after administering the pharmaceutical composition comprising the anti-CD3 immunotoxin to the subject.

4. The method according to claim 1, further comprising transplanting a solid organ, tissue or cells from the donor into the recipient.

5. The method according to claim 4, wherein the donor solid organ, donor tissue, or donor cells comprise kidney, heart, lung, liver, intestine, pancreas, skin, vascular composite allografts (VCAs), leukocytes, hepatocytes, pancreatic islets, corneal epithelial cells, bone marrow (hematopoietic stem cells, HCT) or a combination thereof.

6. The method according to claim 4, further comprising assessing allo-antibody or anti-donor antibody production in the subject before, during and/or after transplantation of the donor solid organ or tissue into the recipient.

7. The method according to claim 1, further comprising administering an immunosuppressant to the subject.

8. The method according to claim 4, wherein the transplanting of the donor solid organ or donor tissue comprises transplanting the donor solid organ or donor tissue at a time when both T cells and B cells are repressed.

9. (canceled)

10. The method according to claim 1, wherein administering the peripheral donor blood cells to the subject occurs with minimal to no peripheral donor blood cell engraftment.

11. The method according to claim 1, wherein the anti-CD3 immunotoxin comprises a recombinant fusion toxin.

12. The method according to claim 11, wherein the recombinant fusion toxin comprises an anti-human CD3 binding domain and a truncated diphtheria toxin.

13. The method according to claim 12, wherein the anti-human CD3 binding domain comprises an anti-human CD3 epsilon specific monoclonal antibody and the truncated diphtheria toxin comprises a translocation and a catalytic domain of a truncated diphtheria toxin.

14. The method according to claim 7, wherein the immunosuppressant comprises one or more of a steroid, Janus kinase inhibitors, calcineurin inhibitors, mTOR inhibitors, IMDH inhibitors, polyclonal antibodies, and monoclonal antibodies.

15. The method according to claim 14, wherein the immunosuppressant comprises one or more immunosuppressant comprising prednisone (e.g. Deltasone, Orasone), budesonide (Entocort EC), prednisolone (Millipred), tofacitinib (Xeljanz), cyclosporine (Neoral, Sandimmune, SangCya), tacrolimus (Astagraf XL, Envarsus XR, Prograf), sirolimus (Rapamune), everolimus (Afinitor, Zortress), azathioprine (Azasan, Imuran), leflunomide (Arava), mycophenolate (CellCept, Myfortic), abatacept (Orencia), adalimumab (Humira), anakinra (Kineret), certolizumab (Cimzia), etanercept (Enbrel), golimumab (Simponi), infliximab (Remicade), ixekizumab (Taltz), natalizumab (Tysabri), rituximab (Rituxan), secukinumab (Cosentyx), tocilizumab (Actemra), ustekinumab (Stelara), vedolizumab (Entyvio), basiliximab (Simulect), daclizumab or (Zinbryta).

16. The method according to claim 14, wherein the immunosuppressant comprises one or more of tacrolimus, mycophenolate and a steroid.

17. The method according to claim 1, wherein MHC compatibility of donor and recipient is selected from fully MHC-matched, haplo-mismatched, or fully mismatched.

18. The method according to claim 1, 2 of 3, wherein the peripheral donor blood cells are obtained without at least one of cytokine mobilization and stem cell enrichment.

19. (canceled)

20. A pharmaceutical composition comprising, one or more anti-CD3 immunotoxin; and peripheral blood cells obtained from a donor of an organ.

21. (canceled)

22. A kit comprising the pharmaceutical composition according to claim 20; and at least one container.

23. A method for depleting T cells in organs and tissues of a subject comprising administering one or more anti-CD3 immunotoxin to a subject in need of such a treatment.

24. (canceled)

25. The method according to claim 23, wherein the subject has GvHD.

26-28. (canceled)
